(19) United States
(12) Patent Application Publication
(10) Pub. No.: US 2022/0096436 A1
(43) Pub. Date: Mar. 31, 2022

(54) COMBINATION PRODUCT FOR THE TREATMENT OF CANCER

(71) Applicant: Debiopharm International S.A., Lausanne (CH)

(72) Inventors: Sergio Adrian Szyldergemajn Altman, Saint Prex (CH); Grégoire Vuagniaux, Lausanne (CH); Norbert Wiedemann, Lausanne (CH); Daniela Purcea, Lausanne (CH)

(73) Assignee: Debiopharm International S.A., Lausanne (CH)

(21) Appl. No.: 17/423,370

(22) PCT Filed: Jan. 17, 2020
(86) PCT No.: PCT/EP2020/051179

§ 371 Date: Jul. 15, 2021

(30) Foreign Application Priority Data
Jan. 17, 2019 (EP) 19152384.4

(51) Int. Cl. A61K 31/407; A61P 35/00; A61K 45/06

(52) U.S. Cl. CPC A61K 31/407; A61P 35/00; A61K 2039/505; A61K 2300/00; A61K 45/06

(57) ABSTRACT

Methods of administering a therapeutically effective amount of Debio 1143, or another IAP antagonist, and a therapeutically effective amount of nivolumab, for the treatment of cancer, are provided.

### TECHNICAL FIELD

The present invention relates to a combination product for use in the treatment of cancer. In particular, the present invention relates to combinations of (5S,8S,10aR)-N-benzhydryl-5-((S)-2-(methylamino)propanamido)-3-(3 -methylbutanoyl)-6-oxodecahydropyrrolo[1,2-a][1,5]diazocine-8-carboxamide (also known as Debio 1143, AT-406, and SM-406) with a specific immune checkpoint inhibitor, nivolumab, for the treatment of patients with cancer. Further aspects of the present invention relate to combinations of IAP antagonists other than Debio 1143 with nivolumab for the treatment of cancer.

### BACKGROUND ART

Resistance of tumor cells to apoptosis is a major problem in current cancer treatment. It is recognized that dysfunction of the apoptosis machinery is a hallmark of cancer. Defects in this machinery result in apoptosis resistance and make current anticancer therapies less effective or ineffective. The aggressive cancer cell phenotype is the result of multiple genetic and epigenetic alterations leading to deregulation of intracellular signaling pathways. Future efforts towards the development of new, molecular target-specific anticancer therapies must include new strategies that specifically target resistance of cancer cells to apoptosis.

IAPB are a class of key regulators of apoptosis characterized by the presence of one to three protein domains known as BIR. cIAP1 and cIAP2 play a critical role in the regulation of death receptor-mediated apoptosis and NFκ-B signaling pathways, which drive the expression of genes relevant for inflammation and immunity; XIAP is a central regulator of both death receptor-mediated and mitochondria-mediated apoptosis pathways. XIAP and cIAP1/2 play key roles in cancer cell resistance to a variety of anticancer drugs, and thus are promising drug targets.

Smac released from mitochondria, is an endogenous inhibitor of XIAP, cIAP1, cIAP2, and ML-IAP. Its amino-terminal tetrapeptide Ala-Val-Pro-Ile binds to a well-defined surface groove in the BIR-3 domain of XIAP. Moreover, Smac proteins can form a homodimer, interacting with both XIAP BIR-3 and BIR-2 domains to release both initiator and effector caspases to promote apoptosis.

A series of monovalent and bivalent Smac mimetics were designed and synthesized to mimic either one or two tetrapeptide Ala-Val-Pro-Ile Smac binding motifs. Both types of Smac mimetics show high binding affinities to XIAP, cIAP1/2. These Smac mimetics also show excellent activity against tumor cells, both inducing apoptosis and inhibiting cell growth and have the potential to promote anti-tumor immunity in combination with immune-oncology agents through NFκ-B signaling modulation.

Debio 1143 is a monovalent, orally available, small molecule antagonist of IAPB that has demonstrated potent single-agent anti-tumor activity in multiple models of human cancer, i.e. of the bladder, breast, head and neck, lung, ovary, pancreas, and prostate.

Immune checkpoints are regulators of immune activation. An example of such a regulator comprises programmed cell death protein 1 (PD-1) and programmed death-ligand 1 (PD-L1). PD-1 is expressed on the surface of T cells whereas PD-L1 is expressed on the surface of many more cells. Binding of PD-L1 to the PD-1 receptor inhibits TCR-mediated activation of IL-2 production and T cell proliferation.

Cancer cells have been known to overexpress PD-L1 to evade the host's immune system. Thus, PD-L1/PD-1 inhibitors have been touted as a possible therapy against cancer. Anti-PD-1 antibodies have been considered to be more promising for the treatment of cancer (You et al., 2018. J Cancer. 9(7):1200-1206). Further, recently phase III trials of Nivolumab for the treatment of both squamous and non-squamous non-small cell lung cancer (NSCLC), alone or in combination with chemotherapy succeed in showing statistically significant overall survival improvement in patients with both PD-L1 positive and negative tumors, leading to regulatory approval in the US, EU, Japon, Canada and other countries (Brahmer J et al NEJM 2015; Borghaei et al. NEJM 2015).

Beug et al., 2014. Oncoimmunology. 3: e28541 suggests that the combination of various immunotherapies with Smac mimetics could result in effective cancer therapies. However, the combination of Debio 1143 with nivolumab is not disclosed, hinted or suggested.

WO 2016/054555 A2 discloses different combination therapies for the treatment of cancer. In some embodiments, the publication suggests combining an IAP inhibitor with an anti-PD-1 or anti-PD-L1 antibody. In particular, LCL-161 is named as a possible IAP inhibitor and it is suggested that LCL-161 should be administered once a week or once every two weeks, albeit no data is provided. Further, WO 2016/054555 A2 provides mouse model data of LCL-161 in combination with an anti-PD-1 antibody. WO 2016/054555 A2 does not disclose Debio 1143 and its combination with an anti-PD-L1 nor does WO 2016/054555 A2 provide any data where the combination of an IAP inhibitor with nivolumab is tested.

WO 2017/143449 A1 also discloses different combination therapies for the treatment of cancer. In some embodiments, the publication suggests combining an IAP inhibitor with an immune checkpoint inhibitor such as an anti-PD-1 or anti-PD-L1 antibody. Mouse model data alleging the efficacy of LCL-161 in combination with an anti-PD-1 antibody for the treatment of cancer is also provided. The combination of Debio 1143 with nivolumab is not disclosed and no data is provided for the combination of an IAP inhibitor with nivolumab.

WO 2019/077132 A1 describes combination therapies of cancer patients including Debio 1143 with immune checkpoint inhibitors and especially anti-PD-L1 antibodies like avelumab. The content of this patent application is incorporated herein in its entirety.

None of the herein cited prior art documents provide any data wherein the combination of Debio 1143 with an anti-PD-1 antibody is tested. Further, none of the prior art documents have tested the efficacy of the combination of Debio 1143 with an anti-PD-1 antibody in humans.

Therapies targeting PD-1 and IAPB separately have shown anti-tumor effects in pre-clinical studies and in the clinic, but improving their anti-tumor efficacy and the proportion of responders remain important goals. Accordingly, there remains a need to develop novel therapeutic options for the treatment of cancers. In particular, there exists a need for methods of treating cancer that improve the efficacy of Debio 1143 or anti-PD-1 antibodies in one or more types of cancer. The present invention provides a combination product for use in the treatment of cancer to address the above need.

### FIGURES

FIG. 1: Anti-tumor activity of Debio 1143, anti-PD-1 antibody, and their combination in the subcutaneous B16F10 mouse melanoma syngeneic model. (A) Effect of Debio 1143 dose on anti-tumor efficacy of the combination. (B) Effect of Debio 1143 dose schedule on anti-tumor efficacy of the combination.

### SUMMARY OF THE INVENTION

Without limiting the scope of the present invention, it is hypothesized that the combination of Debio 1143 or another IAP antagonist with the anti-PD-1 antibody nivolumab can target a cancer cell through the following mechanisms:
1) The anti-PD-1 antibody blocks the PD-1/PD-L1 axis which allows for signaling of the TCR of a CD8+ T-cell with its associated antigen presented by the cancer cell through a MHC-I molecule. Concurrent depletion of the IAPB through Debio 1143 or other IAP antagonist treatment enhances T-cell activation, likely by providing a tumor necrosis factor receptor superfamily (TNFRSF) co-stimulatory response (similar to 4-1BB or OX40 activation), resulting in enhanced activation and expansion of tumor-specific CD8+ T-cells. As a result, Granzyme B (GrzB) and Perforin are secreted to kill target cells.
2) Debio 1143-mediated antagonism of the casp-3 inhibitor, XIAP, results in enhanced death of tumor cells by GrzB. The same effect may be expected for other IAP antagonists.
3) The depletion of cIAP1 and cIAP2 by Debio 1143 or other IAP antagonist leads to increased local production of TNF-α by T-cells in the tumor microenvironment, an effect that is likely mediated by activation of the alternative NFκB pathway.
4) As a result of cIAP1/2 loss, Debio 1143-treated cancer cells or cancer cells treated by another IAP antagonist are sensitized to cell death induction in the presence of proinflammatory cytokines, such as TNF-α.

Potentiation may be additive, or it may be synergistic. The potentiating effect of the combination therapy is at least additive. The present inventors have surprisingly found that the combination of Debio 1143 with an anti-PD-1 antibody results in an improved treatment.

Earlier studies suggested that, unlike LCL-161 which is usually administered once or twice a week (WO 2016/054555 A2: p 14, lines 4-5), Debio 1143 is more effective in combination therapies when administered more frequently (see Example 3 of WO 2019/077132 A1). Thus, in the ongoing clinical trials, Debio 1143 is administered for 10 consecutive days.

Thus, the present invention provides combination products and pharmaceutical compositions, which comprise Debio 1143 or another IAP antagonist and the anti-PD-1 antibody nivolumab, that are suitable for treating cancer.

The present invention also provides methods of administering a combination product comprising Debio 1143 and nivolumab or alternatively comprising another IAP antagonist and nivolumab. The nivolumab and Debio 1143, or alternatively nivolumab and another IAP antagonist, can be administered in a first-line, second-line or subsequent treatment line of the cancer. In some embodiments, the cancer is resistant to prior cancer therapy. In certain embodiments, the method is for treating a human patient having cancer comprising administering to the patient, in need thereof, a therapeutically effective amount of Debio 1143 or of another IAP antagonist and a therapeutically effective amount of nivolumab. In some embodiments, the nivolumab is administered intravenously (e.g., as an intravenous infusion) or subcutaneously. Preferably, nivolumab is administered as an intravenous infusion. More preferably, the inhibitor is administered for 20-80 minutes, most preferably as a 30 minute or 60 minute intravenous infusion. In some embodiments, nivolumab is administered at a dose of about 240 mg every other week (i.e., every two weeks, or “Q2W”) or about 480 mg every four weeks. In other embodiments, nivolumab and Debio 1143 are used in combination with chemotherapy (CT), radiotherapy (RT) or chemoradiotherapy (CRT).

Provided herein is also a pharmaceutical composition comprising nivolumab, Debio 1143 or another IAP antagonist and at least a pharmaceutically acceptable carrier, diluent, excipient and/or adjuvant. Nivolumab and Debio 1143 or another IAP antagonist can be provided in a single or separate unit dosage forms. Separate unit dosage forms are preferred since the preferred mode of administration for Debio 1143 and most other IAP anatagonists is oral administration while nivolumab is preferably administered by intravenous infusion. The pharmaceutical composition may be for use as a medicament, particularly for use in a method of treating cancer.

Also provided herein is nivolumab in combination with Debio 1143 or another IAP antagonist for use as a medicament, particularly for use in a method of treating cancer. Similarly, Debio 1143 is or another IAP antagonist provided in combination with nivolumab for use as a medicament, particularly for use in a method of treating cancer. Also provided is a combination product comprising nivolumab and Debio 1143 or another IAP antagonist for any purpose, for use as a medicament or in a method of treating cancer. The combination of nivolumab and Debio 1143 or another IAP antagonist can be provided in a single or separate unit dosage forms with separate dosage forms being preferred. Also provided is the use of a combination product for the manufacture of a medicament for the treatment of cancer, comprising nivolumab and Debio 1143 or another IAP antagonist.

Also provided is a composition comprising nivolumab for use in a method of treating cancer, wherein the composition is administered in combination with Debio 1143 or another IAP antagonist. Similarly, a composition comprising Debio 1143 or another IAP antagonist for use in a method of treating cancer, wherein the composition is administered in combination with nivolumab is also provided. Either composition can be a pharmaceutical composition further comprising a pharmaceutically acceptable carrier, diluent, excipient and/or adjuvant.

### DETAILED DESCRIPTION OF THE INVENTION

### Definitions

The following definitions are provided to assist the reader. Unless otherwise defined, all terms of art, notations, and other scientific or medical terms or terminology used herein are intended to have the meanings commonly understood by those of skill in the chemical and medical arts. In some cases, terms with commonly understood meanings are defined herein for clarity and/or for ready reference, and the inclusion of such definitions herein should not be construed as representing a substantial difference over the definition of the term as generally understood in the art.

In some embodiments, the term “about” refers to a deviation of ±10% from the recited value. When the word “about” is used herein in reference to a number, it should be understood that still another embodiment of the invention includes that number not modified by the presence of the word “about”.

“Administering” or “administration of” a drug to a patient (and grammatical equivalents of this phrase) refers to direct administration, which may be administration to a patient by a medical professional or may be self-administration, and/or indirect administration, which may be the act of prescribing a drug. E.g., a physician who instructs a patient to self-administer a drug or provides a patient with a prescription for a drug is administering the drug to the patient.

“Antibody” is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact polyclonal or monoclonal antibodies, but also, unless otherwise specified, any antigen-binding fragment or antibody fragment thereof that competes with the intact antibody for specific binding, fusion proteins comprising an antigen-binding portion (e.g., antibody-drug conjugates), any other modified configuration of the immunoglobulin molecule that comprises an antigen recognition site, antibody compositions with poly-epitopic specificity, and multi-specific antibodies (e.g., bispecific antibodies). However, intact, i.e. non-fragmented, monoclonal antibodies are preferred.

The term “cancer” refers to a group of diseases, which can be defined as any abnormal benign or malignant new growth of tissue that possesses no physiological function and arises from uncontrolled usually rapid cellular proliferation and has the potential to invade or spread to other parts of the body. Non-limiting examples include: Acute granulocytic leukemia, Acute lymphocytic leukemia, Acute myelogenous leukemia, Adenocarcinoma, Adrenal cancer, Anaplastic astrocytoma, Angiosarcoma, Appendix cancer, Astrocytoma, Basal cell carcinoma, B-Cell lymphoma, Bile duct cancer, Bladder cancer, Bone cancer, Bone marrow cancer, Bowel cancer, Brain cancer, Brain stem glioma, Brain tumor, Breast cancer, Carcinoid tumors, Cervical cancer, Cholangiocarcinoma, Chondrosarcoma, Chronic lymphocytic leukemia, Chronic myelogenous leukemia, Colon cancer, Colorectal cancer, Craniopharyngioma, Cutaneous lymphoma, Cutaneous melanoma, Diffuse astrocytoma, Ductal carcinoma in situ, Endometrial cancer, Ependymoma, Epithelioid sarcoma, Esophageal cancer, Ewing sarcoma, Extrahepatic bile duct cancer, Eye cancer, Fallopian tube cancer, Fibrosarcoma, Gallbladder cancer, Gastric cancer, Gastrointestinal cancer, Gastrointestinal carcinoid cancer, Gastrointestinal stromal tumors, Germ cell tumor, Glioblastoma multiforme, Glioma, Hairy cell leukemia, Head and neck cancer, Hemangioendothelioma, Hodgkin lymphoma, Hypopharyngeal cancer, Infiltrating ductal carcinoma, Infiltrating lobular carcinoma, Inflammatory breast cancer, Intestinal Cancer, Intrahepatic bile duct cancer, Invasive/infiltrating breast cancer, Islet cell cancer, Jaw cancer, Kaposi sarcoma, Kidney cancer, Laryngeal cancer, Leiomyosarcoma, Leptomeningeal metastases, Leukemia, Lip cancer, Liposarcoma, Liver cancer, Lobular carcinoma in situ, Low-grade astrocytoma, Lung cancer, Lymph node cancer, Lymphoma, Male breast cancer, Medullary carcinoma, Medulloblastoma, Melanoma, Meningioma, Merkel cell carcinoma, Mesenchymal chondrosarcoma, Mesenchymous, Mesothelioma, Metastatic breast cancer, Metastatic melanoma, Metastatic squamous neck cancer, Mixed gliomas, Mouth cancer, Mucinous carcinoma, Mucosal melanoma, Multiple myeloma, Mycosis Fungoides, Myelodysplastic Syndrome, Nasal cavity cancer, Nasopharyngeal cancer, Neck cancer, Neuroblastoma, Neuroendocrine tumors, Non-Hodgkin lymphoma, Non-small cell lung cancer, Oat cell cancer, Ocular cancer, Ocular melanoma, Oligodendroglioma, Oral cancer, Oral cavity cancer, Oropharyngeal cancer, Osteogenic sarcoma, Osteosarcoma, Ovarian cancer, Ovarian epithelial cancer, Ovarian germ cell tumor, Ovarian primary peritoneal carcinoma, Ovarian sex cord stromal tumor, Pancreatic cancer, Papillary carcinoma, Paranasal sinus cancer, Parathyroid cancer, Pelvic cancer, Penile cancer, Peripheral nerve cancer, Peritoneal cancer, Pharyngeal cancer, Pheochromocytoma, Pilocytic astrocytoma, Pineal region tumor, Pituitary gland cancer, Primary central nervous system lymphoma, Prostate cancer, Rectal cancer, Renal cell carcinoma, Renal pelvis cancer, Rhabdomyosarcoma, Salivary gland cancer, Sarcoma, Bone sarcoma, Soft tissue sarcoma, Uterine sarcoma, Sinus cancer, Skin cancer, Small cell lung cancer, Small intestine cancer, Spinal cancer, Spinal column cancer, Spinal cord cancer, Spinal tumor, Squamous cell carcinoma, Stomach cancer, Synovial sarcoma, T-cell lymphoma, Testicular cancer, Throat cancer, Thymoma/thymic carcinoma, Thyroid cancer, Tongue cancer, Tonsil cancer, Transitional cell cancer, Triple-negative breast cancer, Tubal cancer, Tubular carcinoma, Urethral cancer, Uterine adenocarcinoma, Uterine cancer, Vaginal cancer and Vulvar cancer.

In preferred embodiments, the present invention concerns the treatment of cancer, wherein the term “cancer” refers to cancer that fulfills one or more of the following criteria:
(i) the cancer is an advanced, unresectable and/or metastatic solid malignancy and/or cancer;
(ii) the cancer is selected from the group consisting of
   a. small cell lung cancer (SCLC);
   b. squamous cell carcinoma of the head and neck (SCCHN);
   c. GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers;
   d. platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD).
(iii) The cancer has previously been treated with at least one prior line of standard systemic chemotherapy in the advanced/unresectable cancer setting.
(iv) The cancer is platinum resistant, relapsed, or refractory or platinum sensitive.
(v) The cancer has progressed or relapsed during or after a prior anti-programmed cell death-1 (PD-1)/programmed cell death-ligand 1 (PD-L1)-based treatment, given either as a single agent or in combination with standard/approved chemotherapy, tyrosine kinase inhibitors (TKIs), radiotherapy (RT) or other monoclonal antibodies (mAbs) that are not known to modulate/inhibit immune checkpoints (CPIs).

The invention relates in particular to the treatment of cancers that fulfill two or more, preferably three or more, more preferably all of the above criteria (i) to (v). All disclosures hereinabove and herein below concerning the combination therapies, drug combinations, combination products, pharmaceutical compositions, treatment methods, etc. of the present invention apply especially to these specific cancer types, as defined under one or more of items (i) to (v) above

The term “combination product” can refer to (i′) a product comprised of two or more regulated components that are physically, chemically, or otherwise combined or mixed and produced as a single entity; (ii′) two or more separate products packaged together in a single package or as a unit and comprised of drug and device products, device and biological products, or biological and drug products; (iii′) a drug, device, or biological product packaged separately that according to its investigational plan or proposed labeling is intended for use only with an approved individually specified drug, device, or biological product where both are required to achieve the intended use, indication, or effect and where upon approval of the proposed product the labeling of the approved product would need to be changed, e.g., to reflect a change in intended use, dosage form, strength, route of administration, or significant change in dose; or (iv′) any investigational drug, device, or biological product packaged separately that according to its proposed labeling is for use only with another individually specified investigational drug, device, or biological product where both are required to achieve the intended use, indication, or effect.

“Combination therapy”, “in combination with” or “in conjunction with” as used herein denotes any form of concurrent, parallel, simultaneous, sequential or intermittent treatment with at least two distinct treatment modalities (i.e., compounds, components, targeted agents or therapeutic agents). As such, the terms refer to administration of one treatment modality before, during, or after administration of the other treatment modality to the subject. The modalities in combination can be administered in any order. The therapeutically active modalities are administered together (e.g., simultaneously in the same or separate compositions, formulations or unit dosage forms) or separately (e.g., on the same day or on different days and in any order as according to an appropriate dosing protocol for the separate compositions, formulations or unit dosage forms) in a manner and dosing regimen prescribed by a medical care taker or according to a regulatory agency. In general, each treatment modality will be administered at a dose and/or on a time schedule determined for that treatment modality. Optionally, three or more modalities may be used in a combination therapy. Additionally, the combination therapies provided herein may be used in conjunction with other types of treatment. For example, other anti-cancer treatment may be selected from the group consisting of chemotherapy, surgery, radiotherapy (radiation) and/or hormone therapy, amongst other treatments associated with the current standard of care for the subject.

A “complete response” or “complete remission” or “CR” indicates the disappearance of all target lesions as defined in the RECIST v1.1 guideline. This does not always mean the cancer has been cured.

The term “Debio 1143”, “AT-406”, or “SM-406” refers to (5S,8S,10aR)-N-benzhydryl-5-((S)-2-(methylamino)propanamido)-3-(3 -methylbutanoyl)-6- oxodecahydropyrrolo[1,2-a][1,5]diazocine-8-carboxamide (CAS Registry Number: 1071992-99-8) and/or pharmaceutically acceptable salts thereof. Preferably, the free base form of Debio 1143 is used in any aspect of the present invention. Its synthesis has been described previously (Cai et al., 2011. J Med Chem. 54(8):2714-26 and WO 2008/128171—Example 16).

“Disease free survival” (DFS) refers to the length of time during and after treatment that the patient remains free of disease.

“Dose” and “dosage” refer to a specific amount of active or therapeutic agents for administration.

Such amounts are included in a “dosage form,” which refers to physically discrete units suitable as unitary dosages for human subjects and other mammals, each unit containing a predetermined quantity of active agent calculated to produce the desired onset, tolerability, and therapeutic effects, in association with one or more suitable pharmaceutical excipients such as carriers.

The terms “GCIG CA-125 criteria” or “GCIG-Rustin-modified criteria” refer to the criteria that should be used to define progression-free survival and response to treatment using the serum marker CA-125 and agreed by the Gynecological Cancer intergroup (GCIG) (Rustin et al., int J Gynecol Cancer. 2011; 21(2):419-23). Patients are scored as having attained a CA-125 response if they meet the GCIG-Rustin-modified criteria which require that there is at least a 50% reduction in CA-125 levels from a pre-treatment sample.

The term “IAP Antagonist” is used herein to characterize a substance that is capable of inhibiting, blocking, slowing or reducing the function of TAP proteins. It is used herein to have the same meaning as “IAP Inhibitor”. IAP proteins are proteins that regulate (inhibit) apoptosis. They are characterized by the presence of at least one BIR domain such as XIAP, cIAP1, cIAP2, Cp-IAP, NAIP, and Op-TAP. IAP proteins are described for instance in J. Silke and P. Meier, Cold Spring Harb Perspect Biol 2013; 5:a008730 and references cited therein. TAP antagonists in the sense of the present invention are substances capable of inhibiting at least one of these TAP proteins, preferably two or more TAP proteins and most preferably cIAP1 and/or cIAP2. The Smac (Diablo) protein is an endogenous antagonist of TAP proteins. TAP antagonists are therefore in some instances referred to as Smac mimetics. Such Smac mimetics are meant to be encompassed by the term “IAP antagonist”. However, the present invention can also be successfully practiced with IAP antagonists that are not Smac mimetics, e.g. because they have a clearly different structure. There is an interaction between IAP antagonists and the BIR3 domain of IAP proteins. For the purpose of the present invention, it is of particular interest that an interaction between the IAP antagonists and cIAP1 and/or cIAP2 leads to degradation of these proteins and subsequent NF-κB modulation. An TAP antagonist may be identified as a compound having a Kᵢ of <1 μM against XIAP BIR3, cIAP1 BIR3 and/or cIAP2 BIR3, when carrying out the experiment underlying FIG. 4 of the publication by Cai et al. in J Med Chem. 2011, 54(8):2714-26.

The terms “individual”, “patient” or “subject” are used interchangeably in the present application and are not meant to be limiting in any way. The “individual”, “patient” or “subject” can be of any age, sex and physical condition. Preferably, the methods of treatment and combination products of the present invention are for use in a human patient. In other words, the individual, patent or subject is preferably human.

“Infusion” or “infusing” refers to the introduction of a drug-containing solution into the body through a vein for therapeutic purposes. Generally, this is achieved via an intravenous bag.

The term “iRECIST” refer to guideline, criteria, or standard, describes a standard approach to solid tumor measurement and definitions for objective assessment of change in tumor size for use in adult and pediatric cancer clinical trials testing immunotherapeutics and published in Seymour et al, Lancet Oncol. 2017; 18(3). The term iRECIST refers to the novel modified RECIST v1.1 guideline for immunotherapeutics. Patients with asymptomatic, but radiologically observed PD as per iRECIST, may continue the study treatment, until PD is confirmed as per iRECIST or symptoms occurs or the Investigator/patient decision to withdraw treatment, whichever occurs first. Under iRECIST the response can be iUPD, before iCR, iPR, or iSD. “i” indicates immune responses assigned using iRECIST. RECIST=Response Evaluation Criteria in Solid Tumors. iUPD=unconfirmed progression. iCPD=confirmed progression. iCR=complete response. iPR=partial response. iSD=stable disease.

“Nivolumab” is a fully human immunoglobulin G4 (IgG4) monoclonal antibody, which binds to the PD-1 receptor and blocks its interaction with PD-L1 and PD-L2. It is marketed by Bristol-Myers Squibb as a concentrate for infusion under the trade name Opdivo®. Details of the authorized formulation, medical indications and administration are provided in the SmPC at https://www.ema.europa.eu/documents/product-information/opdivo-epar-product-information_en.pdf. Further information on nivolumab is found in the corresponding Wikipedia entry under https://en.wikipedia.org/wiki/Nivolumab (version of Jan. 16, 2018) and references cited therein.

“Overall Survival” (OS) refers to the time from patient enrollment to death or censored at the date last known alive. OS includes a prolongation in life expectancy as compared to naive or untreated individuals or patients. Overall survival refers to the situation wherein a patient remains alive for a defined period of time, such as one year, five years, etc., e.g., from the time of diagnosis or treatment.

A “partial response” or “PR” refers to at least a 30% decrease in the sum of diameters of target lesions, taking as reference the baseline sum diameter, in response to treatment, as defined in the RECIST v1.1 guideline.

The term “pharmaceutically acceptable adjuvant” refers to any and all substances which enhance the body's immune response to an antigen. Non-limiting examples of pharmaceutically acceptable adjuvants are: Alum, Freund's Incomplete Adjuvant, MF59, synthetic analogs of dsRNA such as poly(I:C), bacterial LPS, bacterial flagellin, imidazolquinolines, oligodeoxynucleotides containing specific CpG motifs, fragments of bacterial cell walls such as muramyl dipeptide and Quil-A®.

As used herein, “pharmaceutically acceptable carrier” or “pharmaceutically acceptable diluent” means any and all solvents, dispersion media, coatings, antibacterial and antifungal agents, isotonic and absorption delaying agents, compatible with pharmaceutical administration. The use of such media and agents for pharmaceutically active substances is well known in the art. Acceptable carriers, excipients, or stabilizers are nontoxic to recipients at the dosages and concentrations employed and, without limiting the scope of the present invention, include: additional buffering agents; preservatives; co-solvents; antioxidants, including ascorbic acid and methionine; chelating agents such as EDTA; metal complexes (e.g., Zn-protein complexes); biodegradable polymers, such as polyesters; salt-forming counterions, such as sodium, polyhydric sugar alcohols; amino acids, such as alanine, glycine, glutamine, asparagine, histidine, arginine, lysine, ornithine, leucine, 2-phenylalanine, glutamic acid, and threonine; organic sugars or sugar alcohols, such as lactitol, stachyose, mannose, sorbose, xylose, ribose, ribitol, myoinisitose, myoinisitol, galactose, galactitol, glycerol, cyclitols (e.g., inositol), polyethylene glycol; sulfur containing reducing agents, such as urea, glutathione, thioctic acid, sodium thioglycolate, thioglycerol, [alpha]-monothioglycerol, and sodium thio sulfate; low molecular weight proteins, such as human serum albumin, bovine serum albumin, gelatin, or other immunoglobulins; and hydrophilic polymers, such as polyvinylpyrrolidone. Other pharmaceutically acceptable carriers, excipients, or stabilizers, such as those described in Remington's Pharmaceutical Sciences 16th edition, Osol, A. Ed. (1980) may also be included in a pharmaceutical composition described herein, provided that they do not adversely affect the desired characteristics of the pharmaceutical composition. Pharmaceutical compositions comprising Debio 1143 preferably comprise Starch 1500 (reference to quality standard: Ph. Eur. 01/2010:1267) as a pharmaceutically acceptable excipient.

“Platinum-based therapy” refers to any therapy which involves the use of platinum-based agents, such as cisplatin, carboplatin and oxaliplatin for the treatment of cancer. Platinum-based agents are alkylating agents which bind covalently to DNA and cross-links DNA strands, resulting in inhibition of DNA synthesis and function as well as inhibition of transcription. Platinum-based chemotherapy combinations have demonstrated improvements over single-agent therapy in advanced NSCLC (see Dubey & Schiller, 2004. Hematol Oncol Clin N Am. 18:101-114). Thus, in some embodiments, the platinum-based therapy is a platinum-based doublet chemotherapy (Du & Morgensztern, 2015. Cancer J. 21(5):366-370). According to current guidelines, the first-line treatment strategy for advanced NSCLC should take into account age, histology, molecular pathology, comorbidities, and the performance status of patients, and platinum-based doublet chemotherapy (PT-DC) has been recommended as the standard first-line treatment for such individuals, especially those without epidermal growth factor receptor (EGFR) mutations (Hu et al., 2016. Medicine (Baltimore). 95(28):e4183).

The term “platinum-based therapy cycle” refers to a course of treatment that is repeated on a regular schedule with periods of rest in between. For example, treatment given for one week followed by three weeks of rest is one treatment cycle.

“Platinum-resistant” is defined as relapse or progressive disease (PD) occurring within 1 to 6 months (180 days) after a platinum-containing chemotherapy.

“Progressive disease” or “disease that has progressed” refers to the appearance of one more new lesions or tumors and/or the unequivocal progression of existing non-target lesions as defined in the RECIST v1.1 guideline. Progressive disease or disease that has progressed can also refer to a tumor growth of more than 20 percent since treatment began, either due to an increase in mass or in spread of the tumor.

“Progression free survival” (PFS) refers to the time from enrollment to disease progression or death. PFS is generally measured using the Kaplan-Meier method and Response Evaluation Criteria in Solid Tumors (RECIST) 1.1 standards. Generally, progression free survival refers to the situation wherein a patient remains alive, without the cancer getting worse. PFS based on CA-125 will be defined as the time from first study drug infusion until the GCIG-Rustin-modified criteria of progression are met, or until the date of death (with or without disease progression). Duration of CA-125 response will be defined as the time between when the CA-125 was first documented to have decreased by 50% in a patient who meets all the GCIG-Rustin-modified criteria for a CA-125 response, and the time the CA-125 is first documented to have risen to the point where the patient meets GCIG criteria of disease progression.

The term “RECIST” means Response Evaluation Criteria in Solid Tumours. RECIST guideline, criteria, or standard, describes a standard approach to solid tumor measurement and definitions for objective assessment of change in tumor size for use in adult and pediatric cancer clinical trials. RECIST v1.1 means version 1.1 of the revised RECIST guideline and it is published in European Journal of Cancers 45 (2009) 228-247.

The term “respond favorably” generally refers to causing a beneficial state in a subject. With respect to cancer treatment, the term refers to providing a therapeutic effect on the subject. Positive therapeutic effects in cancer can be measured in a number of ways (See, Weber, 2009. J Nucl Med. 50 Suppl 1:1S-10S). For example, tumor growth inhibition, molecular marker expression, serum marker expression, and molecular imaging techniques can all be used to assess therapeutic efficacy of an anti-cancer therapeutic. With respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. A favorable response can be assessed, for example, by increased progression-free survival (PFS), disease-free survival (DFS), or overall survival (OS), complete response (CR), partial response (PR), or, in some cases, stable disease (SD), a decrease in progressive disease (PD), a reduced time to progression (TTP) or any combination thereof.

“Stable disease” refers to disease without progression or relapse as defined in the RECIST v1.1 guideline. In stable disease there is neither sufficient tumor shrinkage to qualify for partial response, nor sufficient tumor increase to qualify as progressive disease.

The term “therapeutically effective amount” refers to an amount of Debio 1143 or another TAP antagonist, and/or nivolumab which has a therapeutic effect and which is able to treat cancer. In the case of cancer, e.g., an advanced solid malignancy, the therapeutically effective amount of the drug can reduce the number of cancer cells; reduce the tumor size or burden; inhibit (i.e., slow to some extent and in a certain embodiment, stop) cancer cell infiltration into peripheral organs; inhibit (i.e., slow to some extent and in a certain embodiment, stop) tumor metastasis; inhibit, to some extent, tumor growth; relieve to some extent one or more of the symptoms associated with the cancer; and/or result in a favorable response such as increased progression-free survival (PFS), disease-free survival (DFS), or overall survival (OS), complete response (CR), partial response (PR), or, in some cases, stable disease (SD), a decrease in progressive disease (PD), a reduced time to progression (TTP) or any combination thereof. To the extent the drug can prevent growth and/or kill existing cancer cells, it can be cytostatic and/or cytotoxic. A “prophylactically effective amount” refers to an amount effective, at dosages and for periods of time necessary, to achieve the desired prophylactic result. Typically but not necessarily, since a prophylactic dose is used in subjects prior to or at an earlier stage of disease, the prophylactically effective amount will be less than the therapeutically effective amount.

“Time to Tumor Progression” (TTP) is defined as the time from enrollment to disease progression. TTP is generally measured using the RECIST v1.1 criteria.

The terms “treatment” and “therapy”, as used in the present application, refer to a set of hygienic, pharmacological, surgical and/or physical means used with the intent to cure and/or alleviate a disease and/or symptoms with the goal of remediating the health problem. The terms “treatment” and “therapy” include preventive and curative methods, since both are directed to the maintenance and/or reestablishment of the health of an individual or animal. Regardless of the origin of the symptoms, disease and disability, the administration of a suitable medicament to alleviate and/or cure a health problem should be interpreted as a form of treatment or therapy within the context of this application.

“Unit dosage form” as used herein refers to a physically discrete unit of therapeutic formulation appropriate for the subject to be treated. It will be understood, however, that the total daily usage of the compositions of the present invention will be decided by the attending physician within the scope of sound medical judgment. The specific effective dose level for any particular subject or organism will depend upon a variety of factors including the disorder being treated and the severity of the disorder; activity of specific active agent employed; specific composition employed; age, body weight, general health, sex and diet of the subject; time of administration, and rate of excretion of the specific active agent employed; duration of the treatment; drugs and/or additional therapies used in combination or coincidental with specific compound(s) employed, and like factors well known in the medical arts.

PFS, DFS, and OS can be measured by standards set by the National Cancer Institute and the U.S. Food and Drug Administration for the approval of new drugs. See Johnson et al., (2003) J Clin. Oncol. 21(7):1404-1411.

The present invention relates to combinations of Debio 1143 or another IAP antagonist with nivolumab in combination therapies, combination products, pharmaceutical compositions containing drug combinations, kits containing such drug combinations in separate containers, pharmaceutical compositions containing one of these drugs for use in combination with the respective other drug (and vice versa) as well as methods of treatment comprising the administration of at least one of these products. Unless the context dictates otherwise, all references to any one of the above-mentioned aspects of the present invention should also be understood as references to the other aspects of the present invention as listed above. For instance, references to the method of the present invention should also be understood as disclosures of pharmaceutical compositions of the present invention that are to be used in these methods. Likewise, references to the pharmaceutical compositions of the present invention should also be understood as disclosures of methods of the present invention using these pharmaceutical compositions.

Although the present invention is described hereinbelow primarily by describing “specific embodiments” of the invention (or using similar terminology such as “certain embodiments”, etc.), such disclosures of multiple embodiments should also be understood as disclosures of the respective combinations of features, unless the context dictates otherwise.

### IAP Antagonist

The main aspect of the present invention concerns the combination of Debio 1143 with nivolumab and the medical uses described herein of this combination. However, according to other aspects of the present invention, it is possible to use IAP antagonists other than Debio 1143 instead of Debio 1143.

In its broadest aspect, the present invention may be implemented using any IAP antagonist that falls into the scope of the above definition. In more specific preferred aspects, the IAP antagonist may be selected from:
monovalent IAP antagonists other than Debio 1143, such as LCL-161 (Novartis, CAS No.: 1005342-46-0) and CUDC 427/GDC 0917 (Curis/Genentec, CAS No 1446182-94-0);
bivalent IAP antagonists such as TL-32711/Birinapant (Medivir, CAS No.: 1260251-31-7), AZD5582 (AstraZeneca; CAS No. 1258392-53-8) and APG-1387 (Ascentage Pharma, SM-1387, CAS No. 1570231-89-8); and
further IAP antagonists such as ASTX660 (Astex, CAS No. 1799328-86-1), SBP-0636457 (Sandford Burnham Prebys Medical Discovery Institute, CAS No. 1422180-49-1) and JP1201 (Joyant Pharmaceuticals).

The relevant information provided herein for the main aspect inventive concept, such as mode of action, dosage, formulation, administration schedule, etc., apply in a suitably adapted form to the alternative IAP antagonists that can be used in the present invention. For instance, the IAP antagonist birinapant can be advantageously be administered by IV infusion (please see clinicaltrials.gov, studies NCT02288208 and NCT01681368). By consequence, the aspect of the present invention relating to a combination of birinapant with nivolumab is preferably implemented by administering birinapant by IV administration (whereas, by contrast, Debio 1143 is described herein as being preferably administered orally).

Further information on suitable dosages, administration forms, etc. for other IAP antagonists can be found in the literature, such as WO 2017/143449 A and D. Finlay et al. in F1000Research 2017, 6(F1000 Faculty Rev):587 (https://doi.org/10.12688/f1000research.10625.1) and references cited therein or, more specifically,
US 2013/005663 and WO 2016/054555 A for LCL-161;
US 2013/0005663 A for CUDC 427;
WO 2014/121178 A and US 2014/243276 A for birinapant;
WO 2010/142994 A for AZD5582;
Z. Chen et al. in Front Pharmacol. 2018; 9: 1298; doi: 10.3389/fphar.2018.01298 as well as B. Li et al. in J Exp Clin Cancer Res. 2018 March 12; 37(1):53. doi: 10.1186/s13046-018-0703-9 and literature cited therein for APG-1387;
EP 3 083 616 A for ASTX660;
WO 2014/085489 A for SBP-0636457; and
WO 2011/059763 A for JP1201.

Dosage indications in the literature for monotherapy or combination therapy with IAP inhibitors may be adopted as described in the literature, e.g. as cited above. Alternatively, suitable dosages may be determined by means of dose escalation studies. This latter option allows to take changes in dosage requirements due to combination effects with nivolumab into account.

### Methods of Use and Pharmaceutical Compositions

The present invention provides a combination product comprising Debio 1143 or another IAP antagonist and nivolumab for use in a method of treating cancer.

The present invention also provides a composition comprising Debio 1143 or another IAP antagonist for use in a method of treating cancer comprising administering nivolumab. Alternatively, the present invention provides nivolumab for use in a method of treating cancer comprising administering Debio 1143 or another IAP antagonist.

The present invention also provides methods of administering a combination product comprising Debio 1143 or another IAP antagonist and nivolumab. Further, the present invention provides methods of administering Debio 1143 or another IAP antagonist and nivolumab. In certain embodiments, the method is for treating a human patient having cancer comprising administering to the patient, in need thereof, a therapeutically effective amount of Debio 1143 or another IAP antagonist and a therapeutically effective amount of nivolumab.

In certain embodiments, the method for treating cancer is a method for treating a human patient having cancer comprising administering to the patient, in need thereof, a therapeutically effective amount of Debio 1143 or another IAP antagonist and a therapeutically effective amount of nivolumab.

The following information applies specifically to the main aspect relating to the combination of nivolumab with Debio 1143. However, This information may be used as a starting point for developing suitable therapies for the combinations of nivolumab with other IAP antagonists according to further aspects of the invention.

In some embodiments, the therapeutically effective amount of Debio 1143 is about 75 to about 250 mg per day. Preferably, the therapeutically effective amount of Debio 1143 is about 75-100, 75-125, 75-150, 75-175, 75-200, 75-225, 100-125, 100-150, 100-175, 100-200, 100-225, 125-150, 125-175, 125-200, 125-225, 150-175, 150-200, 150-225, 175-200, 175-225 or 200-225 mg per day. In a preferred embodiment, the therapeutically effective amount of Debio 1143 is 100, 150 or 200 mg per day. Generally, all indications of the amount of Debio 1143 provided herein refer to the total amount of Debio 1143 administered per day.

In some embodiments, Debio 1143 is administered orally. In some embodiments, Debio 1143 is administered in capsular form or tablet form. In some embodiments, Debio 1143 is administered orally as a capsule containing 75, 100, 125, 150, 175, 200, 225 or 250 mg Debio 1143 and especially 75 mg, 100 mg, 150 mg or 200 mg Debio 1143. In some embodiments, Debio 1143 is administered orally as a tablet containing 75, 100, 125, 150, 175, 200, 225 or 250 mg Debio 1143 and especially 100, 150 mg or 200 mg Debio 1143. Preferably, Debio 1143 is administered orally as a capsule containing 200 mg Debio 1143.

In certain embodiments, the therapeutically effective amount of Debio 1143 is administered as one dose one time per day. In certain embodiments, the therapeutically effective amount of Debio 1143 is divided into multiple doses that are administered as multiple doses two, three, or four times per day.

In some embodiments, Debio 1143 is administered daily for 10 consecutive days. In some embodiments, Debio 1143 is administered once daily for 10 consecutive days. In some embodiments, the method of treatment comprises a 28 day cycle comprising administering Debio 1143 for 10 consecutive days, followed by administering no Debio 1143 for 4 consecutive days, followed by administering Debio 1143 for 10 consecutive days, followed by administering no Debio 1143 for 4 consecutive days.

As noted above, nivolumab is a fully human immunoglobulin G4 (IgG4) monoclonal antibody, which binds to the PD-1 receptor and blocks its interaction with PD-L1 and PD-L2. When PD-L1 that is found on tumor cells binds to PD-1 that is found on immune system cells, the PD-1 signaling pathway is activated, inhibiting an immune response. By blocking this interaction, nivolumab allows the immune system to recognize and attack tumor cells. In two pivotal phase I trials in advanced solid tumors, nivolumab showed significant clinical anti-tumor activity and was relatively well tolerated at 10 mg/kg once every 2 weeks (q2w) as monotherapy or in combination with other agents (non-CPIs). (Brahmer J R, Drake CG, Wollner I, Powderly J D, Picus J, Sharfman W H, et al. Phase I study of single-agent anti-programmed death-1 (MDX-1106) in refractory solid tumors: safety, clinical activity, pharmacodynamics, and immunologic correlates. J Clin Oncol. 2010; 28(19):3167-75; Topalian S L, Hodi F S, Brahmer J R, Gettinger S N, Smith D C, McDermott D F, et al. Safety, activity, and immune correlates of anti-PD-1 antibody in cancer. The New England journal of medicine. 2012; 366(26):2443-54.) This led to several randomized phase III trials of nivolumab monotherapy in advanced cancers including melanoma, non-small cell lung cancer (NSCLC), renal cell carcinoma (RCC), classical Hodgkin lymphoma, squamous cell carcinoma of the head and neck (SCCHN) and others. (Antonia S J, Lopez-Martin J A, Bendell J, Ott P A, Taylor M, Eder J P, et al. Nivolumab alone and nivolumab plus ipilimumab in recurrent small-cell lung cancer (CheckMate 032): a multicentre, open-label, phase 1/2 trial. Lancet Oncol. 2016; 17(7):883-95). In the pooled dataset of all patients enrolled in these phase III trials and receiving nivolumab 3-10 mg/kg q2w as monotherapy across tumor types (n=2,578) with minimum follow-up ranging from 2.3 to 28 months, the most frequent adverse reactions (>10%) were fatigue (30%), rash (17%), pruritus (13%), diarrhea (13%), and nausea (12%). The majority of adverse reactions were mild to moderate (Grade 1 or 2). With a minimum of 24 months follow-up in NSCLC, no new safety signals were identified. (European Medicine Agency. Nivolumab-Annex I SUMMARY OF PRODUCT CHARACTERISTICS. 2018. p. 1-80; Champiat S, Lambotte O, Barreau E, Belkhir R, Berdelou A, Carbonnel F, et al. Management of immune checkpoint blockade dysimmune toxicities: a collaborative position paper. Annals of oncology: official journal of the European Society for Medical Oncology. 2016; 27(4):559-74).

Efficacy of nivolumab monotherapy was assessed in a recent meta-analysis that included 27 worldwide clinical trials (n=5,551). The pooled objective response rate (ORR), the 6-month PFS and the 1-year OS rates were 26% (95% confidence interval (CI) 21-31), 40% (95% CI 34-46) and 52% (95% CI: 43-62), respectively. (Tie Y, Ma X, Zhu C, Mao Y, Shen K, Wei X, et al. Safety and efficacy of nivolumab in the treatment of cancers: A meta-analysis of 27 prospective clinical trials. Int J Cancer. 2017; 140(4):948-58). In Europe, nivolumab (OPDIVO®) as monotherapy or in combination with ipilimumab is currently indicated for the treatment of advanced melanoma, NSCLC, RCC, classical Hodgkin's lymphoma, SCCHN and urothelial carcinoma. Due to antibody-specific kinetics, a flat dose was derived from the 3 mg/kg q2w dose by multiplying for an average 80 kg weight, thus based on the current prescribing information, a nivolumab infusion at either 240 mg (flat dose) q2w or 480 mg every four weeks (q4w) (starting from Cycle 3) was identified as the recommended phase II dose (RP2D). The two treatment schedules are currently being used in daily oncology practice given the extended terminal half-life of IgG4 antibodies such as nivolumab and the relative lack of potential interactions. (EMA, Nivolumab, SUMMARY OF PRODUCT CHARACTERISTICS).

The therapeutically effective amount is sufficient for treating one or more symptoms of a disease or disorder associated with nivolumab and Debio 1143, or with nivolumab and another IAP antagonist, respectively. In some embodiments that employ nivolumab in the combination therapy, the dosing regimen will comprise administering nivolumab at a dose of about 240 mg at intervals of about 14 days (±2 days) or at a dose of about 480 mg at intervals of about 28 days (±2 days) throughout the course of treatment. In some embodiments, nivolumab is administered intravenously. In some embodiments, nivolumab is administered on days 1 and 15 of a 28-day cycle. In certain embodiments, nivolumab is administered intravenously for about 30 minutes at a dose of about 240 mg every two weeks. In another embodiment, the nivolumab dose will be 480 mg administered as 1-hour intravenous infusions every 28 days. In some embodiments, a time window of minus 10 minutes and plus 20 minutes is permitted. In other embodiments, no significant variation of infusion time is permitted.

Nivolumab may be administered up to 3 days before or after the scheduled day of administration of each cycle due to administrative reasons.

In some embodiments, the method further comprises administering an antihistamine (anti-H1) and acetaminophen to the patient prior to administering nivolumab. In some embodiments, the antihistamine (anti-H1) and acetaminophen are administered to the patient about 30 minutes to about 60 minutes prior administering the nivolumab. In some embodiments, the antihistamine (anti-H1) and acetaminophen are administered prior to each of the first four administrations of nivolumab. In some embodiments, the antihistamine (anti-H1) is diphenhydramine. In some embodiments, about 25 to about 50 mg diphenhydramine is administered in the method.

In various embodiments, the method of the invention is employed as a first, second, third or later line of treatment. Preferably, the method of the invention is employed as a second, third or later line of treatment. A line of treatment refers to a place in the order of treatment with different medications or other therapies received by a patient. First-line therapy regimens are treatments given first, whereas second- or third-line therapy is given after the first-line therapy or after the second-line therapy, respectively. Therefore, first-line therapy is the first treatment for a disease or condition. In patients with cancer, first-line therapy, sometimes referred to as primary therapy or primary treatment, can be surgery, chemotherapy, radiation therapy, or a combination of these therapies. Typically, a patient is given a subsequent chemotherapy regimen (second- or third-line therapy), either because the patient did not show a positive clinical outcome or only showed a sub-clinical response to a first- or second-line therapy or showed a positive clinical response but later experienced a relapse, sometimes with disease now resistant to the earlier therapy that elicited the earlier positive response.

In another embodiment of the invention, the therapeutic combination of the invention is applied in a later line of treatment, particularly a second-line or higher treatment of the cancer. There is no limitation to the prior number of therapies provided that the subject underwent at least one round of prior cancer therapy, although second line, third line, fourth line and fifth line treatment is preferred. The round of prior cancer therapy refers to a defined schedule/phase for treating a subject with, e.g., one or more immunotherapeutic agents (e.g., nivolumab), chemotherapeutic agents, radiotherapy or chemoradiotherapy, and the subject failed with such previous treatment, which was either completed or terminated ahead of schedule. One reason could be that the cancer was resistant to prior therapy. The addition of Debio 1143 or another IAP antagonist may suppress this mechanism of resistance and restore the effect of the immunotherapy. The set of patients with resistance becomes treatable and show improved responses.

As the mode of action of Debio 1143 or other IAP antagonists is different from that of the anti-PD-1 antibodies, the chances to have enhanced immune-related adverse events (irAE) are small although both agents are targeting the immune system. The absence of overlapping immune features in nonclinical findings or in published clinical results makes the risk low for the combination therapy of the invention to show enhanced adverse events above what is generally observed in the class of PD-1 targeting agents. The identified and potential risks for nivolumab, and for Debio 1143 or other IAP antagonists, in each case as single agent, are considered to represent the potential risks for the combination treatment as well.

The current standard of care (SoC) for treating cancer patients often involves the administration of toxic and old chemotherapy regimens. The SoC is associated with high risks of strong adverse events that are likely to interfere with the quality of life (such as secondary cancers). The toxicity profile of the nivolumab/Debio 1143 combination, or the combination of nivolumab with or other IAP antagonists, seems to be much better than the SoC chemotherapy. In one embodiment, the nivolumab/Debio 1143 combination, or the combination of nivolumab with or other IAP antagonists, may be as effective and better tolerated than SoC chemotherapy in patients with cancer resistant to mono- and/or poly-chemotherapy, radiotherapy or chemoradiotherapy.

The patients to be treated with the methods of the present invention are patients having cancer as defined above and especially cancer fulfilling one or more of the criteria defined under items (i) to (v) above. These patients are preferably patients, who additionally fulfil one or more of the following additional criteria, more preferably two or more, three or more, four or more, five or more and most preferably all of the following criteria:
1. The patient has received prior therapy with a minimum washout periods since prior therapy until treatment start (C1D1) (in cases of more than one prior treatment type, whichever has the longest minimum period applies):
   a. 3 weeks for chemotherapy (6 weeks, specifically for nitrosoureas or mitomycin C containing regimens);
   b. 4 weeks for mAbs (other than previous PD-1/PD-L1), or live vaccines
   c. 3 weeks for prior RT (1 week in case of localized antalgic/hemostatic hypofractionated RT flash)
   d. 2 weeks for TKIs, hormonal therapy, other anti-cancer treatment not previously specified or investigational agents or previous anti-PD-1/PD-L1 mAbs
   e. 4 weeks for any major surgery
   f. Immunosuppressive medication: within 2 weeks, with the exception of intranasal and inhaled corticosteroids or systemic corticosteroids at physiological/replacement doses, which should never exceed 10 mg/d prednisone, or an equivalent corticosteroid
2. Measurable disease according to RECIST v1.1 and/or GCIC criteria (if applicable) and documented PD during or after prior PD-1/PD-L1 based therapy
3. ECOG Performance Status=0 or 1
4. Adequate hematologic, renal and hepatic function:
   a. absolute neutrophil count (ANC)≥1.5×10⁹/L,
   b. platelets≥100×10⁹/L,
   c. hemoglobin≥9.0 g/dL,
   d. AST and ALT≤3×ULN (≤5×ULN if liver metastases are present),
   e. total bilirubin≤1.5×ULN,
   f. serum creatinine≤1.5×ULN,
   g. serum albumin≥30 g/L
5. Participants with known central nervous system (CNS) and/or meningeal involvement are clinically asymptomatic, have completed primary CNS therapy more than 4 weeks before treatment start (such as whole brain RT, stereotactic radiosurgery, or complete surgical resection), and have remained off steroids (including tapering doses) for at least 2 weeks
6. Women of child-bearing potential (WOCBP):
   a. Negative serum pregnancy test at screening;
   b. Agreement to use highly effective contraception methods from study entry and for up to 5 months after the last day of study treatment
   c. Agreement from her male partner to use contraception methods
7. Male patients with WOCBP partners who agree to use highly effective contraception methods from study entry and for up to 5 months after the last day of study treatment
8. Patient has not undergone thoracic or head and neck radiation >30 Gy within the 6 weeks prior to C1D1
9. Patient has not received, in total, more than 3 or 4 lines of prior systemic treatments (including adjuvant or neoadjuvant regimens if relapse within six months prior to C1D1)
10. Patient has no active moderate alcohol consumption of more than 100/140 grams of alcohol per week for female/male patients, respectively
11. Patient has no liver cirrhosis Child-Pugh score B or C
12. Patient has not undergone prior treatment with an anti-CTLA-4 or anti-LAG3 in combination with PD-1/PD-L1 CPI
13. Patient has not undergone prior treatment with SMAC mimetics
14. Patient has not had prior PD-1/PD-L1 discontinuation due to severe immune-related toxicity, not resolved upon adequate steroids/immunosuppressive treatment
15. Patient has no requirement of concomitant treatment with medication selected from immunosuppressive agents such as systemic corticosteroids, TNF inhibitors, etc. In addition, there must not be any administration of any of grapefruit juice and grapefruit-containing products, St. John's Wort (=millepertuis) and St. John's Wort-containing products, Amiodarone, Atorvastatine, Boceprevir, Cannabidiol, Carbamazepine, Clarithromicin, Ciclosporine, Cobicistat, Daclatasvir, Diltiazem, Dronedarone, Dipyridamole, Enzalutamide, Erythromycin, Fidaxomicin, Hydroquinidine, Itraconazole, Ivacaftor, Ketoconazole, Lansoprazole, Ledipasvir, Mirabegron, Propafenone, Quinine, Quinidine, Ranolazine, Retigabine, Rifabutin, Rifampin, Rilpivirine, Ritonavir, Saquinavir, Simeprevir, Telaprevir, Telithromycine, Ticagrelor, Tipranavir, Tolvaptan, Ulipristal, Verapamil, Alfuzosine, Apixaban, Apoxetine, Artemether, Atazanavir, Atorvastatin, Avanafil,

Bosentan, Bromocriptine, Cinacalcet, Ergotamine Dihydrate, Dolutegravir, Domperidone, Ebastine, Efavirenz, Eletriptan, Eplerenone, Ergotamine, Fesoterodine, Fosamprenavir, Halofantrine, Indinavir, Ivabradine, Lopinavir, Lumefantrine, Maraviroc, Mizolastine, Manidipine, Nevirapine, Piperaquine, Praziquantel, Quetiapine, Repaglinide, Rivaroxaban, Rupatadine, Sildenafil, Simvastatine, Sirolimus, Solifenacine, Tacrolimus, Tadalafil, Tamsulosine, Tolterodine, Vardenafil during the treatment or shortly before the treatment of the invention such that an interaction with the drugs of the present invention becomes possible
16. Patient has no history of allergic reactions attributed to compounds of similar chemical or biologic composition to Debio 1143, or other IAP antagonists, or nivolumab or their constituents
17. Patient is not unable to swallow or retain oral medications

In certain embodiments, methods of treating a human patient having cancer, as defined herein, comprise administering to the patient, in need thereof, about 100, 150 or 200 mg/day of Debio 1143 according to one of the following schedules, together with about 240 mg nivolumab every 14 days or about 480 mg nivolumab every 28 days:
(i) 4 days administration followed by 3 days of no administration;
(ii) 5 days administration followed by 2 days of no administration;
(iii) 6 days administration followed by 1 day of no administration;
(iv) 8 days administration followed by 6 days of no administration;
(v) 9 days administration followed by 5 days of no administration;
(vi) 10 days administration followed by 4 days of no administration;
(vii) 11 days administration followed by 3 days of no administration;
(viii) 12 days administration followed by 2 days of no administration;
(ix) 13 days administration followed by 1 day of no administration;
(x) daily administration with no interruptions. In some circumstances du to medical reasons, administration of any of the drugs may be withheld temporarily. In some embodiments, the method of treatment comprises a 28 day cycle comprising a sequence (a)-(b)-(a)-(b) of the following stages:
(a) administering Debio 1143 for 9, 10 or 11 consecutive days; and
(b) administering no Debio 1143 for 5, 4 or 3 consecutive days, respectively.

In certain embodiments, methods of treating a human patient having advanced, unresectable and/or metastatic cancer comprising administering to the patient about 100, 150 or 200 mg of Debio 1143 daily for 10 days followed by no administration for 4 days together with about 240 mg nivolumab every 14 days or about 480 mg nivolumab every 28 days are provided herein. In some embodiments, the patient with advanced, unresectable and/or metastatic cancer previously received platinum-based therapy. In some embodiments, the patient is orally administered Debio 1143. In some embodiments, Debio 1143 is provided in capsular form. In some embodiments, the patient is orally administered

Debio 1143 for 10 consecutive days.

In some embodiments, the method of treatment comprises administering Debio 1143 for 10 consecutive days followed by 4 consecutive days wherein Debio 1143 is not administered.

Debio 1143 is more effective in combination therapies when administered more frequently (see Example 2). Thus, administering Debio 1143 for 10 consecutive days should be more effective than administering Debio 1143 less frequently, for example, once or twice a week. Further, the time period of e.g. four consecutive days in which no Debio 1143 is administered follows the ten consecutive days of treatment to ensure that the patient can recover from the treatment.

In some embodiments, the nivolumab is administered once every two weeks. In some embodiments, the nivolumab is administered on days 1 and 15 of a 28-day cycle. In some embodiments, the nivolumab is administered intravenously. In some embodiments, the method comprises administering an antihistamine (anti-H1) and acetaminophen to the patient prior to administering the nivolumab. In some embodiments, the antihistamine (anti-H1) and acetaminophen are administered to the patient about 30 minutes to about 60 minutes prior to administering the nivolumab. In some embodiments, the antihistamine (anti-H1) and acetaminophen are administered prior to each of the first four administrations of nivolumab. In some embodiments, the antihistamine (anti-H1) is diphenhydramine. In some embodiments, about 25 to about 50 mg diphenhydramine is administered.

In certain embodiments, methods of treatment comprising at least one 28 day cycle comprising stages (a)-(b)-(c)-(d):
(a) administering Debio 1143 for a first 10 consecutive day period;
(b) administering no Debio 1143 for a first 4 consecutive day period;
(c) administering Debio 1143 for a second 10 consecutive day period; and
(d) administering no Debio 1143 for a second 4 consecutive day period

are provided herein. In some embodiments, the nivolumab is administered once every two weeks. In some embodiments, the nivolumab is administered on days 1 and 15 of the 28-day cycle. In some other embodiments, the nivolumab is administered only on day 1 of the 28-day cycle. In some embodiments, the nivolumab is administered intravenously. In some embodiments, the method further comprises administering an antihistamine (anti-H1) and acetaminophen to the patient prior to administering the nivolumab. In some embodiments, the antihistamine (anti-H1) and acetaminophen are administered to the patient about 30 minutes to about 60 minutes prior to administering the nivolumab. In some embodiments, the antihistamine (anti-H1) and acetaminophen are administered prior to each of the first four administrations of nivolumab. In some embodiments, the antihistamine (anti-H1) is diphenhydramine. In some embodiments, about 25 to about 50 mg diphenhydramine is administered.

In certain embodiments, the method of treatment comprises a 28-day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period;
(e) administering the nivolumab on day 1 of the 28-day cycle; and
(f) administering the nivolumab on day 15 of the 28-day cycle.

In certain embodiments, methods of treating a human patient having Small Cell Lung Cancer (SCLC) comprising orally administering to the patient, in need thereof, about 100 mg, 150 mg or about 200 mg of Debio 1143 and intravenously about 240 mg nivolumab, wherein the method of treatment comprises a 28 day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period;
(e) administering the nivolumab on day 1 of the 28-day cycle; and
(f) administering the nivolumab on day 15 of the 28-day cycle are provided herein.

In certain other embodiments, methods of treating a human patient having Small Cell Lung Cancer comprising orally administering to the patient, in need thereof, about 100, 150 mg or about 200 mg of Debio 1143 and intravenously about 480 mg nivolumab, wherein the method of treatment comprises a 28 day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period; and
(e) administering the nivolumab on day 1 of the 28-day cycle; are provided herein.

By following the above treatment protocol, an effective treatment of SCLC can be achieved.

In certain embodiments, methods of treating a human patient having squamous cell carcinoma of the head and neck (SCCHN) comprising orally administering to the patient, in need thereof, about 100, 150 mg or about 200 mg of Debio 1143 and intravenously about 240 mg nivolumab, wherein the method of treatment comprises a 28 day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period;
(e) administering the nivolumab on day 1 of the 28-day cycle; and
(f) administering the nivolumab on day 15 of the 28-day cycle are provided herein.

In certain other embodiments, methods of treating a human patient having squamous cell carcinoma of the head and neck (SCCHN) comprising orally administering to the patient, in need thereof, about 100, 150 mg or about 200 mg of Debio 1143 and intravenously about 480 mg nivolumab, wherein the method of treatment comprises a 28 day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period; and
(e) administering the nivolumab on day 1 of the 28-day cycle; are provided herein.

By following the above treatment protocol, an effective treatment of squamous cell carcinoma of the head and neck (SCCHN) can be achieved.

In certain embodiments, methods of treating a human patient having GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers comprising orally administering to the patient, in need thereof, about 100, 150 mg or about 200 mg of Debio 1143 and intravenously about 240 mg nivolumab, wherein the method of treatment comprises a 28 day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period;
(e) administering the nivolumab on day 1 of the 28-day cycle; and
(f) administering the nivolumab on day 15 of the 28-day cycle are provided herein.

In certain other embodiments, methods of treating a human patient having GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers comprising orally administering to the patient, in need thereof, about 100, 150 mg or about 200 mg of Debio 1143 and intravenously about 480 mg nivolumab, wherein the method of treatment comprises a 28 day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period; and
(e) administering the nivolumab on day 1 of the 28-day cycle; are provided herein.

By following the above treatment protocol, an effective treatment of GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers can be achieved.

In certain embodiments, methods of treating a human patient having platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD) comprising orally administering to the patient, in need thereof, about 100, 150 mg or about 200 mg of Debio 1143 and intravenously about 240 mg nivolumab, wherein the method of treatment comprises a 28 day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period;
(e) administering the nivolumab on day 1 of the 28-day cycle; and
(f) administering the nivolumab on day 15 of the 28-day cycle are provided herein.

In certain other embodiments, methods of treating a human patient having platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD) comprising orally administering to the patient, in need thereof, about 100 mg, about 150 mg or about 200 mg of Debio 1143 and intravenously about 480 mg nivolumab, wherein the method of treatment comprises a 28 day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period; and
(e) administering the nivolumab on day 1 of the 28-day cycle; are provided herein.

By following the above treatment protocol, an effective treatment of platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD) can be achieved.

Debio 1143 is preferably administered to the patient having an empty stomach (i.e. patients will fast for 2 h before dosing and for at least 1 h after dosing). It is preferable that the patient takes Debio 1143 at approximately the same time each day (±60 min, more preferably ±30 min).

In certain embodiments, in addition to administering Debio 1143, or another IAP antagonist, and nivolumab, the method of treatment further comprises administering an antihistamine (anti-H1) (e.g., diphenhydramine) and/or acetaminophen to the patient. In some embodiments, the method further comprises administering an antihistamine (anti-H1) to the patient prior to administering the nivolumab. In certain embodiments, the method further comprises administering acetaminophen to the patient prior to administering the nivolumab. In some embodiments, the method further comprises administering an antihistamine (anti-H1) and acetaminophen to the patient prior to administering the nivolumab. In certain embodiments, the antihistamine (anti-H1) is administered to the patient about 30 minutes to about 60 minutes prior to administering the nivolumab. In certain embodiments, the acetaminophen is administered to the patient about 30 minutes to about 60 minutes prior to administering the nivolumab. In certain embodiments, the antihistamine (anti-H1) and acetaminophen are administered to the patient about 30 minutes to about 60 minutes prior to administering the nivolumab. In certain embodiments, the antihistamine (anti-H1) is diphenhydramine. In certain embodiments, about 25 to about 50 mg diphenhydramine is administered. In certain embodiments, the therapeutically effective amount of Debio 1143, or another IAP antagonist, is administered as one dose one time per day. In certain embodiments, the therapeutically effective amount of Debio 1143, or another IAP antagonist, is divided into multiple doses that are administered as multiple doses two, three, or four times per day.

In some embodiments, the platinum-based therapy comprised administering one of more platinum-based agents selected from the group consisting of cisplatin, carboplatin, and oxaliplatin. In some embodiments, the patient has relapsed or progressed after being administered the platinum-based therapy but before being administered Debio 1143, or another IAP antagonist. In some embodiments, the patient previously underwent at least one platinum-based therapy cycle. In some embodiments, the patient previously underwent at least two, three, four, five or six platinum-based therapy cycles. In some embodiments, the platinum-based therapy was stopped after at least one cycle because the disease progressed despite the platinum-based therapy. In some embodiments, the platinum-based therapy was stopped after at least one cycle due to toxicity, wherein the toxicity is associated with the platinum-based therapy.

In some embodiments, the therapeutically effective amount of Debio 1143, or another IAP antagonist, and nivolumab is administered to a patient with an increased expression level of PD-L1. In some embodiments, the PD-L1 expression level is measured by immunohistochemistry (IHC). Immunohistochemistry with anti-PD-L1 primary antibodies can be performed on serial cuts of formalin fixed and paraffin embedded specimens from patients treated with nivolumab and Debio 1143, or another IAP antagonist. In some embodiments, at least 1% of the cells exhibit PD-L1 expression. Preferably, at least 1% of the cancer cells exhibit PD-L1 expression.

This disclosure also provides a kit for determining if the combination of the invention is suitable for therapeutic treatment of a cancer patient, comprising means for determining a protein level of PD-L1, or the expression level of its RNA, in a sample isolated from the patient and instructions for use. In another aspect, the kit further comprises nivolumab for immunotherapy or Debio 1143. In one aspect of the invention, the determination of a high PD-L1 level indicates increased PFS or OS when the patient is treated with the therapeutic combination of the invention. In one embodiment of the kit, the means for determining the PD-L1 peptide level are antibodies with specific binding to PD-L1, respectively.

In some embodiments, the combination product is a pharmaceutical combination product and further comprises a pharmaceutically acceptable carrier, diluent, excipient and/or adjuvant. In some embodiments, nivolumab and/or Debio 1143, or another IAP antagonist, is comprised within one or more pharmaceutical compositions further comprising a pharmaceutically acceptable carrier, diluent, excipient and/or adjuvant.

In one embodiment, nivolumab is a sterile, clear, and colorless solution intended for IV administration. The contents of the nivolumab vials are non-pyrogenic, and do not contain bacteriostatic preservatives.

Nivolumab is formulated as a 10 mg/mL solution and is supplied in single-use glass vials of 4 mL, 10 mL or 24 mL, stoppered with a rubber septum and sealed with an aluminum-based flip-off seal. For administration purposes, nivolumab may be diluted with 0.9% sodium chloride (normal saline solution) or 5% glucose solution. Tubing with in-line, low protein binding 0.2-1.2 micron filter made of polyether sulfone (PES) is used during administration.

In some embodiments, the method of treatment results in a decrease of at least one grade of the Eastern Cooperative Oncology Group Performance Status (ECOG-PS) scale in comparison to the ECOG-PS grade before the start of the method of treatment if the grade before the start of the method of treatment is 4 or less, preferably 2 or less. The evaluation criteria for the ECOG-PS scale are well known in the art (see Oken et al., 1982. Am J Clin Oncol. 5(6):649-55).

In some embodiments, the method of treatment results in a decrease in size of a cancer-associated lesion compared to the size of the lesion before the start of the method of treatment. In some embodiments, the method of treatment results in a decrease in size of at least 30% or higher of a cancer lesion compared to the size of the same lesion before the start of the method of treatment. The size of the lesion can be determined by performing a computed tomography (CT), or magnetic resonance imaging scan of the patient or clinically if applicable (i.e skin lesions). For gynecological cancers, in particular ovarian cancer, GCIG-Rustin-modified criteria apply and CA-125 level decreases by 50%.

In a further aspect, nivolumab and Debio 1143, or another IAP antagonist, are administered sequentially in either order or substantially simultaneously. In some embodiments, the combination regimen comprises the steps of: (a) under the direction or control of a physician, the subject receiving nivolumab prior to first receipt of Debio 1143, or another IAP antagonist; and (b) under the direction or control of a physician, the subject receiving Debio 1143, or another IAP antagonist. In some embodiments, the combination regimen comprises the steps of: (a) under the direction or control of a physician, the subject receiving Debio 1143, or another IAP antagonist, prior to first receipt of nivolumab; and (b) under the direction or control of a physician, the subject receiving nivolumab. In some embodiments, the combination regimen comprises the steps of: (a) prescribing the subject to self-administer, and verifying that the subject has self-administered, nivolumab prior to first administration of Debio 1143, or another IAP antagonist; and (b) administering Debio 1143, or another IAP antagonist, to the subject. In some embodiments, the combination regimen comprises the steps of: (a) prescribing the subject to self-administer, and verifying that the subject has self-administered, Debio 1143, or another IAP antagonist, prior to first administration of nivolumab; and (b) administering nivolumab to the subject. In some embodiments, the combination regimen comprises, after the subject has received nivolumab prior to the first administration of Debio 1143, or another IAP antagonist, administering Debio 1143, or another IAP antagonist, to the subject. In some embodiments, the combination regimen comprises, after the subject has received Debio 1143, or another IAP antagonist, prior to first administration of nivolumab, administering nivolumab to the subject.

Also provided herein is nivolumab for use as a medicament in combination with Debio 1143, or another IAP antagonist. Similarly provided is Debio 1143, or another IAP antagonist, for use as a medicament in combination with nivolumab. Also provided is nivolumab for use in the treatment of cancer in combination with Debio 1143, or another IAP antagonist. Similarly provided is Debio 1143, or another IAP antagonist, for use in the treatment of cancer in combination with nivolumab. Also provided is a combination product comprising nivolumab and Debio 1143, or another IAP antagonist, for use as a medicament. Also provided is the use of a combination product for the manufacture of a medicament for the treatment of cancer, comprising nivolumab and Debio 1143, or another IAP antagonist. The aforementioned combinations and combination products are provided in a single or separate unit dosage forms.

In a further aspect, the invention relates to a kit comprising nivolumab and a package insert comprising instructions for using nivolumab in combination with Debio 1143, or another IAP antagonist, to treat or delay progression of a cancer in a subject. Also provided is a kit comprising Debio 1143, or another IAP antagonist, and a package insert comprising instructions for using Debio 1143, or another IAP antagonist, in combination with nivolumab to treat or delay progression of a cancer in a subject. Also provided is a kit comprising nivolumab and Debio 1143, or another IAP antagonist, and a package insert comprising instructions for using nivolumab and Debio 1143, or another IAP antagonist, to treat or delay progression of a cancer in a subject. The kit can comprise a first container, a second container and a package insert, wherein the first container comprises at least one dose of a medicament comprising nivolumab, the second container comprises at least one dose of a medicament comprising Debio 1143, or another IAP antagonist, and the package insert comprises instructions for treating a subject for cancer using the medicaments. The first and second containers may be comprised of the same or different shape (e.g., vials, syringes and bottles) and/or material (e.g., plastic or glass). The kit may further comprise other materials that may be useful in administering the medicaments, such as diluents, filters, IV bags and lines, needles and syringes. The instructions can state that the medicaments are intended for use in treating a subject having a cancer that tests positive for PD-L1 expression.

### Items of the Invention

The present invention also comprises the following items:
1. A method for treating a human patient having an advanced or metastatic solid malignancy comprising administering to the patient, in need thereof, a therapeutically effective amount of Debio 1143 and a therapeutically effective amount of nivolumab.
2. The method of item 1, wherein the therapeutically effective amount of Debio 1143 is about 75 to about 250 mg per day.
3. The method of any one of items 1-2, wherein the therapeutically effective amount of Debio 1143 is about 75 mg per day.
4. The method of any one of items 1-2, wherein the therapeutically effective amount of Debio 1143 is about 100 mg per day.
5. The method of any one of items 1-2, wherein the therapeutically effective amount of Debio 1143 is about 150 mg per day.
6. The method of any one of items 1-2, wherein the therapeutically effective amount of Debio 1143 is about 200 mg per day.
7. The method of any one of items 1-2, wherein the therapeutically effective amount of Debio 1143 is about 250 mg per day.
8. The method of any one of items 1-7, wherein Debio 1143 is administered orally.
9. The method of any one of items 1-8, wherein Debio 1143 is administered in capsular form.
10. The method of any one of items 1-9, wherein Debio 1143 is administered orally as a capsule containing 75 mg Debio 1143.
11. The method of any one of items 1-9, wherein Debio 1143 is administered orally as a capsule containing 100 mg Debio 1143.
12. The method of any one of items 1-11, wherein the therapeutically effective amount of Debio 1143 is administered as one dose one time per day.
13. The method of any one of items 1-11, wherein the therapeutically effective amount of Debio 1143 is divided into multiple doses that are administered as multiple doses two, three, or four times per day.
14. The method of any one of items 1-13, wherein Debio 1143 is administered once daily for 10 consecutive days.
15. The method of any one of items 1-13, wherein the method of treatment comprises a 28 day cycle comprising two periods of administering Debio 1143 for 10 consecutive days, followed by administering no Debio 1143 for 4 consecutive days.
16. The method of any one of items 1-15, wherein the therapeutically effective amount of nivolumab is about 240 mg or about 480 mg.
17. The method of item 16, wherein the nivolumab is administered once every two weeks if the dose is 240 mg or once every 4 weeks if the dose is 480 mg.
18. The method of any one of items 16 and 17, wherein the nivolumab is administered on days 1 and 15 of a 28-day cycle.
19. The method of any one of items 1-20, wherein nivolumab is administered intravenously.
20. The method of any one of items 16-19 further comprising administering an antihistamine (anti-Hi) and acetaminophen to the patient prior to administering the nivolumab.
21. The method of item 20, wherein the antihistamine (anti-Hi) and acetaminophen are administered to the patient about 30 minutes to about 60 minutes prior administering the nivolumab.
22. The method of any one of items 20 and 21, wherein the antihistamine (anti-H₁) and acetaminophen are administered prior to each of the first four administrations of nivolumab.
23. The method of any one of items 20-22, wherein the antihistamine (anti-H₁) is diphenhydramine.
24. The method of item 23, wherein about 25 to about 50 mg diphenhydramine is administered.
25. The method of any one of items 1-24, wherein the solid malignancy is one or more selected from the group consisting of small cell lung cancer (SCLC); squamous cell carcinoma of the head and neck (SCCHN); GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers; platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD).
26. The method of any one of items 1-25, wherein the advanced solid malignancy is Small Cell Lung Cancer.
27. The method of any one of items 1-26, wherein the advanced solid malignancy is advanced or metastatic Small Cell Lung Cancer.
28. The method of any one of items 1-27, wherein the patient previously received standard therapy including prior PD-1/PD-L1 for treatment of the advanced solid malignancy.
29. The method of items 1-28, wherein the patient has advanced/unresectable solid cancer.
30. A method of treating a human patient having a solid malignancy that is one or more selected from the group consisting of small cell lung cancer (SCLC); squamous cell carcinoma of the head and neck (SCCHN); GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers; platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD), the method comprising administering to the patient, in need thereof, about 75 mg to about 250 mg daily of Debio 1143 for about 20 days of a 28-day treatment cycle and about 480 mg nivolumab in one or two doses of the 28-day treatment cycle.
31. The method of item 30, wherein the patient is orally administered Debio 1143.
32. The method of item 31, wherein Debio 1143 is provided in capsular form.
33. The method of item 30, wherein the patient is orally administered Debio 1143 daily for 10 consecutive days.
34. The method of item 33, wherein the method of treatment comprises a 28 day cycle comprising
   (a) administering Debio 1143 for 10 consecutive days (ON); and
   (b) administering no Debio 1143 for 4 consecutive days (OFF).
35. The method of any of items 30-34, wherein the nivolumab is administered once every two weeks.
36. The method of item 30 or 36, wherein the nivolumab is administered on days 1 and 15 of a 28-day cycle.
37. The method of item 34, wherein the nivolumab is administered on days 1 and 15 of the 28-day cycle.
38. The method of item 35, wherein the nivolumab is administered intravenously.
39. The method of item 36, wherein the nivolumab is administered intravenously.
40. The method of item 37, wherein the nivolumab is administered intravenously.
41. The method of item 35 further comprising administering an antihistamine (anti-H₁) and acetaminophen to the patient prior to administering the nivolumab.
42. The method of item 36 further comprising administering an antihistamine (anti-H₁) and acetaminophen to the patient prior to administering the nivolumab.
43. The method of item 37 further comprising administering an antihistamine (anti-H₁) and acetaminophen to the patient prior to administering the nivolumab.
44. The method of item 41, wherein the antihistamine (anti-H₁) and acetaminophen are administered to the patient about 30 minutes to about 60 minutes prior to administering the nivolumab.
45. The method of item 42, wherein the antihistamine (anti-H₁) and acetaminophen are administered to the patient about 30 minutes to about 60 minutes prior to administering the nivolumab.
46. The method of item 43, wherein the antihistamine (anti-H₁) and acetaminophen are administered to the patient about 30 minutes to about 60 minutes prior to administering the nivolumab.
47. The method of item 44, wherein the antihistamine (anti-H₁) and acetaminophen are administered prior to each of the first four administrations of nivolumab.
48. The method of item 45, wherein the antihistamine (anti-H₁) and acetaminophen are administered prior to each of the first four administrations of nivolumab.
49. The method of item 46, wherein the antihistamine (anti-H₁) and acetaminophen are administered prior to each of the first four administrations of nivolumab.
50. The method of item 47, wherein the antihistamine (anti-H₁) is diphenhydramine.
51. The method of item 48, wherein the antihistamine (anti-H₁) is diphenhydramine.
52. The method of item 49, wherein the antihistamine (anti-H₁) is diphenhydramine.
53. The method of item 50, wherein about 25 to about 50 mg diphenhydramine is administered.
54. The method of item 51, wherein about 25 to about 50 mg diphenhydramine is administered.
55. The method of item 52, wherein about 25 to about 50 mg diphenhydramine is administered.
56. The method of item 31, wherein the method of treatment comprises a 28 day cycle comprising
   (a) administering Debio 1143 for a first 10 consecutive day period;
   (b) administering no Debio 1143 for a first 4 consecutive day period;
   (c) administering Debio 1143 for a second 10 consecutive day period; and
   (d) administering no Debio 1143 for a second 4 consecutive day period.
57. The method of item 56, wherein the nivolumab is administered once every two weeks.
58. The method of item 56, wherein the nivolumab is administered on days 1 and 15 of the 28-day cycle.
59. The method of item 57, wherein the nivolumab is administered intravenously.
60. The method of item 58, wherein the nivolumab is administered intravenously.
61. The method of item 59 further comprising administering an antihistamine (anti-H₁) and acetaminophen to the patient prior to administering the nivolumab.
62. The method of item 60 further comprising administering an antihistamine (anti-H₁) and acetaminophen to the patient prior to administering the nivolumab.
63. The method of item 61, wherein the antihistamine (anti-H₁) and acetaminophen are administered to the patient about 30 minutes to about 60 minutes prior to administering the nivolumab.
64. The method of item 62, wherein the antihistamine (anti-H₁) and acetaminophen are administered to the patient about 30 minutes to about 60 minutes prior to administering the nivolumab.
65. The method of item 63, wherein the antihistamine (anti-H₁) and acetaminophen are administered prior to each of the first four administrations of nivolumab.
66. The method of item 64, wherein the antihistamine (anti-H₁) and acetaminophen are administered prior to each of the first four administrations of nivolumab.
67. The method of item 65, wherein the antihistamine (anti-H₁) is diphenhydramine.
68. The method of item 66, wherein the antihistamine (anti-H₁) is diphenhydramine.
69. The method of item 67, wherein about 25 to about 50 mg diphenhydramine is administered.
70. The method of item 68, wherein about 25 to about 50 mg diphenhydramine is administered
71. A method of treating a human patient having a solid malignancy that is one or more selected from the group consisting of small cell lung cancer (SCLC); squamous cell carcinoma of the head and neck (SCCHN); GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers; platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD), the method comprising orally administering to the patient, in need thereof, about 75 mg to about 250 mg per day of Debio 1143 and intravenously about 240 mg nivolumab per administration, wherein the method of treatment comprises a 28 day cycle comprising
   (a) administering Debio 1143 for a first 10 consecutive day period;
   (b) administering no Debio 1143 for a first 4 consecutive day period;
   (c) administering Debio 1143 for a second 10 consecutive day period;
   (d) administering no Debio 1143 for a second 4 consecutive day period;
   (e) administering the nivolumab on day 1 of the 28-day cycle; and
   (f) administering the nivolumab on day 15 of the 28-day cycle.
72. The method of item 71, wherein Debio 1143 is administered in capsular form.
73. The method of item 71 further comprising administering an antihistamine (anti-H₁) to the patient prior to administering the nivolumab.
74. The method of item 71 further comprising administering acetaminophen to the patient prior to administering the nivolumab.
75. The method of item 71 further comprising administering an antihistamine (anti-H₁) and acetaminophen to the patient prior to administering the nivolumab.
76. The method of item 73, wherein the antihistamine (anti-H₁) is administered to the patient about 30 minutes to about 60 minutes prior to administering the nivolumab.
77. The method of item 74, wherein the acetaminophen is administered to the patient about 30 minutes to about 60 minutes prior to administering the nivolumab
78. The method of item 75, wherein the antihistamine (anti-H₁) and acetaminophen are administered to the patient about 30 minutes to about 60 minutes prior to administering the nivolumab.
79. The method of item 76, wherein the antihistamine (anti-H₁) is diphenhydramine
80. The method of item 78, wherein the antihistamine (anti-H₁) is diphenhydramine.
81. The method of item 79, wherein about 25 to about 50 mg diphenhydramine is administered
82. The method of item 80, wherein about 25 to about 50 mg diphenhydramine is administered.
83. The method of any one of items 30-82, wherein the therapeutically effective amount of Debio 1143 is administered as one dose one time per day.
84. The method of any one of items 30-82, wherein the therapeutically effective amount of Debio 1143 is divided into multiple doses that are administered as multiple doses two, three, or four times per day.
85. The method of any one of items 30-84, wherein the patient previously received platinum-based therapy.
86. The method of any one of items 28 and 85, wherein the platinum-based therapy comprised administering one of more platinum-based agents selected from the group consisting of cisplatin, carboplatin, and oxaliplatin.
87. The method of any one of items 28, 85 and 86, wherein the patient has relapsed or progressed after being administered the platinum-based therapy but before being administered Debio 1143.

Other aspects of the invention are listed in itemized form below:
2-1.A method for treating a human patient having an advanced or metastatic solid malignancy comprising administering to the patient, in need thereof, a therapeutically effective amount of an TAP inhibitor other than Debio 1143 and a therapeutically effective amount of nivolumab.
2-2. The method of item 2-1, wherein the TAP inhibitor other than Debio 1143 is administered orally.
2-3. The method of any one of items 2-1 to 2-2, wherein the TAP inhibitor other than Debio 1143 is administered in capsular form.
2-4. The method of any one of items 2-1 to 2-3, wherein the therapeutically effective amount of the IAP inhibitor other than Debio 1143 is administered as one dose one time per day.
2-5. The method of any one of items 2-1 to 2-4, wherein the therapeutically effective amount of the IAP inhibitor other than Debio 1143 is divided into multiple doses that are administered as multiple doses two, three, or four times per day.
2-6. The method of any one of items 2-1 to 2-5, wherein the therapeutically effective amount of nivolumab is about 240 mg or about 480 mg.
2-7. The method of item 2-6, wherein the nivolumab is administered once every two weeks if the dose is 240 mg or once every 4 weeks if the dose is 480 mg.
2-8. The method of any one of items 2-5 and 2-6, wherein the nivolumab is administered on days 1 and 15 of a 28-day cycle.
2-9. The method of any one of items 2-1 to 2-8, wherein nivolumab is administered intravenously.
2-10. The method of any one of items 2-6 to 2-9 further comprising administering an antihistamine (anti-H₁) and acetaminophen to the patient prior to administering the nivolumab.
2-11. The method of item 2-10, wherein the antihistamine (anti-H₁) and acetaminophen are administered to the patient about 30 minutes to about 60 minutes prior administering the nivolumab.
2-12. The method of any one of items 2-10 and 2-11, wherein the antihistamine (anti-H₁) and acetaminophen are administered prior to each of the first four administrations of nivolumab.
2-13. The method of any one of items 2-10-2-12, wherein the antihistamine (anti-H₁) is diphenhydramine.
2-14. The method of item 2-13, wherein about 25 to about 50 mg diphenhydramine is administered.
2-15. The method of any one of items 2-1 to 2-14, wherein the solid malignancy is one or more selected from the group consisting of small cell lung cancer (SCLC); squamous cell carcinoma of the head and neck (SCCHN); GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers; platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD).
2-16. The method of any one of items 2-1 to 2-15, wherein the advanced solid malignancy is Small Cell Lung Cancer.
2-17. The method of any one of items 2-1 to 2-16, wherein the advanced solid malignancy is advanced or metastatic Small Cell Lung Cancer.
2-18. The method of any one of items 2-1 to 2-17, wherein the patient previously received standard therapy including prior PD-1/PD-L1 for treatment of the advanced solid malignancy.
2-19. The method of items 2-1 to 2-18, wherein the patient has advanced/unresectable solid cancer.
2-20. A method of treating a human patient having a solid malignancy that is one or more selected from the group consisting of small cell lung cancer (SCLC); squamous cell carcinoma of the head and neck (SCCHN); GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers;

platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD), the method comprising administering to the patient, in need thereof, about 75 mg to about 250 mg daily of an IAP inhibitor other than Debio 1143 for about 20 days of a 28-day treatment cycle and about 480 mg nivolumab in one or two doses of the 28-day treatment cycle.
2-21. The method of item 2-20, wherein the patient is orally administered the IAP inhibitor other than Debio 1143.
2-22. The method of item 2-21, wherein the IAP inhibitor other than Debio 1143 is provided in capsular form.
2-23. The method of item 2-20, wherein the patient is orally administered Debio 1143 daily for 10 consecutive days.
2-24. The method of any of items 2-20 to 2-23, wherein the nivolumab is administered once every two weeks.
2-25. The method of item 2-20 to 2-24, wherein the nivolumab is administered on days 1 and 15 of a 28-day cycle.
2-26. The method of item 2-25, wherein the nivolumab is administered on days 1 and 15 of the 28-day cycle.
2-27. The method of item 2-24, wherein the nivolumab is administered intravenously.
2-28. The method of item 2-25, wherein the nivolumab is administered intravenously.
2-29. The method of item 2-26, wherein the nivolumab is administered intravenously.
2-30. The method of item 2-24 further comprising administering an antihistamine (anti-H₁) and acetaminophen to the patient prior to administering the nivolumab.
2-31. The method of item 2-25 further comprising administering an antihistamine (anti-H₁) and acetaminophen to the patient prior to administering the nivolumab.
2-32. The method of item 2-25 further comprising administering an antihistamine (anti-H₁) and acetaminophen to the patient prior to administering the nivolumab.
2-33. The method of item 2-30, wherein the antihistamine (anti-H₁) and acetaminophen are administered to the patient about 30 minutes to about 60 minutes prior to administering the nivolumab.
2-34. The method of item 2-31, wherein the antihistamine (anti-H₁) and acetaminophen are administered to the patient about 30 minutes to about 60 minutes prior to administering the nivolumab.
2-35. The method of item 2-32, wherein the antihistamine (anti-H₁) and acetaminophen are administered to the patient about 30 minutes to about 60 minutes prior to administering the nivolumab.
2-36. The method of item 2-33, wherein the antihistamine (anti-H₁) and acetaminophen are administered prior to each of the first four administrations of nivolumab.
2-37. The method of item 2-34, wherein the antihistamine (anti-H₁) and acetaminophen are administered prior to each of the first four administrations of nivolumab.
2-38. The method of item 2-35, wherein the antihistamine (anti-H₁) and acetaminophen are administered prior to each of the first four administrations of nivolumab.
2-39. The method of item 2-36, wherein the antihistamine (anti-H₁) is diphenhydramine.
2-40. The method of item 2-37, wherein the antihistamine (anti-H₁) is diphenhydramine.
2-41. The method of item 2-38, wherein the antihistamine (anti-H₁) is diphenhydramine.
2-42. The method of item 2-39, wherein about 25 to about 50 mg diphenhydramine is administered.
2-43. The method of item 2-40, wherein about 25 to about 50 mg diphenhydramine is administered.
2-44. The method of item 2-41, wherein about 25 to about 50 mg diphenhydramine is administered.
2-45. The method of any one of items 2-20 to 2-44, wherein the therapeutically effective amount of the TAP inhibitor other than Debio 1143 is divided into multiple doses that are administered as multiple doses two, three, or four times per day.
2-46. The method of any one of items 2-20 to 2-45, wherein the patient previously received platinum-based therapy.
2-47. The method of item 2-46, wherein the platinum-based therapy comprised administering one of more platinum-based agents selected from the group consisting of cisplatin, carboplatin, and oxaliplatin.
2-48. The method of any one of items 2-46 and 2-47, wherein the patient has relapsed or progressed after being administered the platinum-based therapy but before being administered the IAP inhibitor other than Debio 1143.

### EXAMPLES

It is understood that the examples and embodiments described herein are for illustrative purposes only and that various modifications or changes in light thereof will be suggested to persons skilled in the art and are to be included within the spirit and purview of this application.

### Example 1: Combination of Debio 1143 with an Anti-CTLA4 Antibody and IDO Inhibitor

The therapeutic efficacy of Debio 1143 combined with an anti-CTLA4 antibody was tested in a TS/A breast cancer mouse syngeneic model. Further, the therapeutic efficacy of Debio 1143 combined with an IDO inhibitor (INCB024360) was tested in a CT-26 colorectal cancer mouse syngeneic model. In both of these cases, the combination therapy did not lead to a statistically significant improvement compared to the respective monotherapies.

Thus, providing a combination therapy comprising Debio 1143 which results in an additive or synergistic effect may require the non-obvious selection of specific immune checkpoint regulator targets. Simply combining Debio 1143 with any immunotherapy is not sufficient to obtain an improved efficacy or an effective combination therapy which can be used to treat any cancer.

### Example 2: Dose-Dependency of Debio 1143 in a Combination Therapy

To test whether Debio 1143 dose-dependently enhanced anti-PD1 anti-tumor activity, the tolerability of oral Debio 1143 at 200 and 300 mg/kg for 5 days/week in combination with 10 mg/kg anti-PD1 twice weekly was tested for a total duration of 2 weeks in tumor-free female C57BL/6 mice. In contrast to the 300 mg/kg dose, combination treatment with the 200 mg/kg dose was well tolerated with only minor body weight loss on treatment and good recovery of all mice upon dosing completion. Thus, 200 mg/kg was chosen as the highest dose level for the efficacy study evaluating the combination in mice bearing s.c. B16F10 melanoma tumors.

C57BL/6 mice were s.c. injected with 2×10⁵ B16F10 tumor cells (see, for example, Bobek et al., 2010. Anticancer Res. 30(12):4799-803) and treatments started at a mean group tumor size of 94 mm³ (n=8/group). Debio 1143 at 100 and 200 mg/kg p.o. was given on 5 days/week in combination with 10 mg/kg i.p. anti-PD1 twice weekly and compared to treatment with vehicle plus isotype antibody. In addition, the 100 mg/kg dose given twice weekly in combination with anti-PD1 at 10 mg/kg i.p. twice weekly was tested.

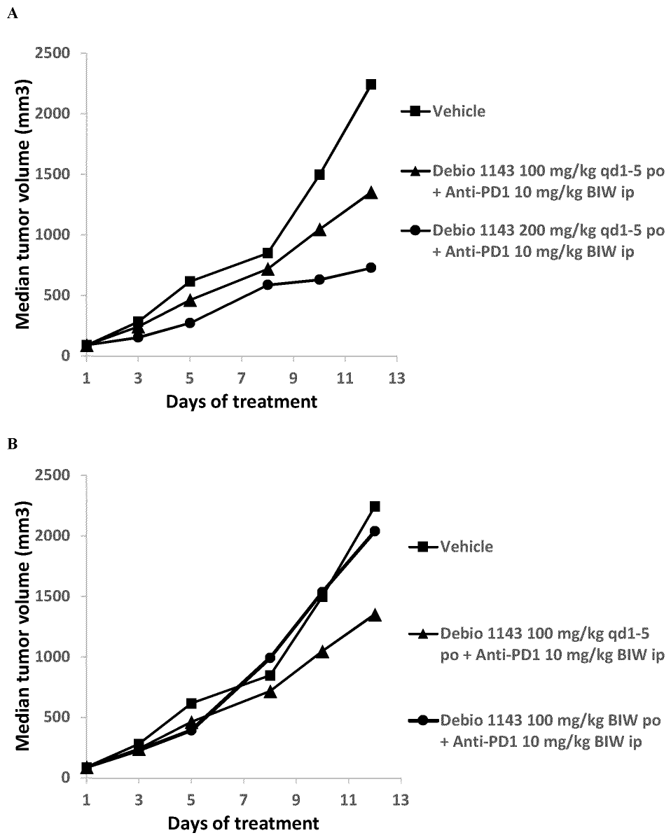

By day 19 from tumor inoculation, treatment with 100 mg/kg Debio 1143 QD1-5 combined with anti-PD-1 at 10 mg/kg also displayed no significant antitumor activity with mean tumor volume of 1049 mm³ (TGI=31%, P=0.155), as compared to the vehicle group (mean tumor volume 1529 mm³; FIG. 1A). However, Debio 1143 at 200 mg/kg in combination with 10 mg/kg anti-PD-1 produced significant antitumor activity (594 mm³; TGI=61%; P=0.005; FIG. 1A). Bi-weekly Debio 1143 at 100 mg/kg (mean tumor volume: 1396 mm³, TGI=9%, P=0.757) in combination with anti-PD-1 at 10 mg/kg also did not produce any antitumor activity as compared to the vehicle group (FIG. 1B). The administration of Debio 1143 at 100 mg/kg over 5 days/week was considered superior to bi-weekly administration for the combination with anti-PD-1 at 10 mg/kg.

### Example 3: Debio 1143 Dosing Trial in Human Patients with Solid Tumors

Debio 1143 in combination with nivolumab is tested in patients with histologically and/or cytologically confirmed advanced/unresectable or metastatic solid tumor, for one of the following indications: small cell lung cancer (SCLC); squamous cell carcinoma of the head and neck (SCCHN);

GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers; platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD).

Part A of this trial is an open-label, multicenter and dose-optimization design applying the classical 3+3 method, aiming at optimizing Debio 1143 dose in combination with standard doses of nivolumab (OPDIVO®) in order to define a safe recommended phase II dose (RP2D) and to assess its safety and feasibility is patients fulfilling the inclusion criteria and exclusion criteria specified below. It involves enrollment of 3-12 patients, the patients having selected advanced/unresectable or metastatic solid tumors, for whom standard therapy has failed, including progression to prior treatments with anti PD-1/PD-L1 given as single agent or in combination with standard/approved chemotherapy, tyrosine kinase inhibitors or other targeted agents, radiotherapy, hormonal therapy or any other non-checkpoint inhibitor monoclonal antibodies, but excluding other antibody or drug specifically targeting T-cell co-stimulation or immune checkpoints pathways (i.e., CTLA-4).

According to the 3+3 dose-optimization design, if none of the three patients at the starting dose cohort experiences a DLT during Cycle 1, three more patients will be treated at the next dose level. However, if one of the first 3 patients experiences a DLT, 3 more patients will be treated at the same dose level. If ≤1 out of 6 evaluable patients experiences a DLT during the first cycle, at the starting dose level, then dose escalation will proceed to the second dose level. If ≥2 DLTs are observed during the first cycle among the 3 or 6 evaluable patients treated with the initial dose level, then recruitment will be stopped temporarily or definitively until the reasons for this finding have been clarified. If the dose is increased to the second dose level, 3 to 6 evaluable patients will be included and the 3+3 design rules will be applied again. If ≤1 out of 6 evaluable patients experiences a DLT during the first cycle at the second dose level, this dose will be considered the optimal dose level. If ≥2 out of 3 or 6 evaluable patients experience a DLT during the first cycle, at the second dose level, then the initial dose level will be declared the optimal dose, given that <33% of evaluable patients have experienced a DLT during the first cycle and there are at least 6 evaluable patients treated at this dose level. Patients within a cohort may all start treatment simultaneously.

The Part B is a multicenter, open-label, basket trial using Debio 1143 in combination with nivolumab at the RP2D, as previously defined in Part A, in patients with selected advanced/unresectable solid tumors. Eligible patients will be simultaneously included, into four cohorts according to tumor type:

Cohort 1: SCLC

Cohort 2: SCCHN

Cohort 3: GI cancers, including esophageal, gastric, colorectal or pancreatobiliary tumors, with known MSI-H/MMRd or other known DDR abnormalities (incl. HRD).

Cohort 4: platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD).

The objective of Part B is to assess whether the combination of Debio 1143 with nivolumab is active overall and in each the cohort. Early futility stopping rules based on objective response (unconfirmed) rate (ORR) will be used. In each cohort, if no unconfirmed response is observed according to Response Evaluation Criteria in Solid Tumors (RECIST v1.1) or Gynecologic Cancer Intergroup (GCIG) criteria (Cohort 4) once the initial 8 evaluable patients have been assessed at least twice after baseline or have discontinued their treatment earlier, futility will be concluded and the recruitment will be stopped in that cohort. If at least one response (unconfirmed) is documented in the initial 8 evaluable patients, recruitment shall continue up to 11 evaluable patients. At least two unconfirmed responses must then be observed in these 11 evaluable patients to continue the recruitment up to 15 evaluable patients in that cohort.

A homogeneity test will be conducted in any non-futile cohorts showing a confirmed response rate of at least 15%. If homogeneous response rates are seen across the cohorts, efficacy data will be pooled and an overall efficacy analysis will be conducted in addition to the analyses by cohort. For the final analysis, first proof of efficacy will be claimed in a given cohort if at least 4 objective responses (confirmed) are reported in the 15 evaluable patients.

In the case of insufficient accrual in any cohort (e.g., less than 2 patients in 6 months) despite concrete measures aiming at increasing the recruitment, enrolment may be stopped in that cohort.

A Data Safety Monitoring Committee (DSMC), composed at least of all actively involved investigators (or their designees), the Sponsor Medical Director and the Contract Research Organization (CRO) medical monitor as voting members, will meet regularly to overview the clinical safety of the patients and laboratory data and the conduct of the study during both study parts A and B. Ad hoc members may be invited upon needed.

During Part A, calls will be scheduled monthly or according to patient recruitment as necessary. A charter will be provided as a separate document.

During Part B the DSMC, will meet approximately every three months, and more frequently if needed in the event of rapid recruitment. A charter will be provided separately.

Patient inclusion criteria are such that patients must fulfill all the following criteria (in both Parts A and B, unless specified otherwise):
1. Willing and able to sign a written informed consent form;
2. Male or female ≥18 years of age.
3. Histologically and/or cytologically confirmed advanced/ unresectable or metastatic solid tumor, for one of the following indications.
   a. SCLC, including extrapulmonary small-cell carcinomas or large cell neuroendocrine lung carcinomas
   b. SCCHN (except nasopharyngeal carcinoma)
   c. GI cancers, including esophageal, gastric, colorectal or pancreatobiliary with known MSI-H/MMRd or any other known DDRs abnormalities, including HRD
   d. Platinum-resistant*EOC, endometrial cancer, PPC or cervical cancer, with known MSI-H/MMRd, hereditary/somatic mutations of the BRCA1 and BRCA2 genes or other known DNA DDRs abnormalities (incl. HRD)
   * Platinum-resistant is defined as relapse or progressive disease (PD) occurring within 1 to 6 months (180 days) after a platinum-containing chemotherapy.
4. Have received at least one prior line of standard systemic chemotherapy in the advanced/unresectable cancer setting (standard adjuvant/neoadjuvant treatment is acceptable if relapse occurred within six months of treatment end)
5. Have progressed or relapsed during or after a prior anti-programmed cell death-1 (PD-1)/programmed cell death-ligand 1 (PD-L1)-based treatment, given either as a single agent or in combination with standard/approved chemotherapy, tyrosine kinase inhibitors (TKIs), radiotherapy (RT) or other monoclonal antibodies (mAbs) that are not known to modulate/inhibit immune checkpoints (CPIs).
6. Minimum washout periods since prior therapy until treatment start (C1D1)(in cases of more than one prior treatment type, whichever has the longest minimum period applies):
   a. 3 weeks for chemotherapy (6 weeks, specifically for nitrosoureas or mitomycin C containing regimens);
   b. 4 weeks for mAbs (other than previous PD-1/PD-L1), or live vaccines
   c. 3 weeks for prior RT (1 week in case of localized antalgic/hemostatic hypofractionated RT flash)
   d. 2 weeks for TKIs, hormonal therapy, other anti-cancer treatment not previously specified or investigational agents or previous anti-PD-1/PD-L1 mAbs
   e. 4 weeks for any major surgery
   f. Immunosuppressive medication: within 2 weeks, with the exception of intranasal and inhaled corticosteroids or systemic corticosteroids at physiological/replacement doses, which should never exceed 10 mg/d prednisone, or an equivalent corticosteroid
7. Measurable disease (Part B only) according to RECIST v1.1 and/or GCIC criteria in Cohort #4 (if applicable) and documented PD during or after prior PD-1/PD-L1 based therapy
8. ECOG Performance Status=0 or 1
9. Adequate hematologic, renal and hepatic function:
   a. absolute neutrophil count (ANC)≥1.5×10⁹/L,
   b. platelets≥100×10⁹/L,
   c. hemoglobin≥9.0 g/dL,
   d. AST and ALT≤3×ULN,
   e. total bilirubin≤1.5×ULN,
   f. serum creatinine≤1.5×ULN (<5×ULN if liver metastases are present),
   g. serum albumin≥30 g/L
10. Available archived tumor samples for biomarker analysis obtained after prior PD-1/PD-L1 treatment failure or, if no archived tumor sample is available, patient must be suitable and willing to undergo a percutaneous or endoscopic biopsy without unacceptable major risks before starting study treatment
11. Participants with known central nervous system (CNS) and/or meningeal involvement will be eligible if they are clinically asymptomatic, have completed primary CNS therapy more than 4 weeks before treatment start (such as whole brain RT, stereotactic radiosurgery, or complete surgical resection), and have remained off steroids (including tapering doses) for at least 2 weeks
12. Women of child-bearing potential (WOCBP):
   a. Negative serum pregnancy test at screening;
   b. Agreement to use highly effective contraception methods from study entry and for up to 5 months after the last day of study treatment
   c. Agreement from her male partner to use contraception methods
13. Male patients with WOCBP partners must agree to use highly effective contraception methods from study entry and for up to 5 months after the last day of study treatment

Exclusion criteria are the following, wherein patients must NOT fulfill any of the following criteria (in both Parts A and B, unless specified otherwise):
14. Thoracic or head and neck radiation >30 Gy within the 6 weeks prior to C1D1
15. Have received, in total, more than 3 (i.e. Cohorts 1&2) or 4 (i.e. Cohorts 3&4) lines of prior systemic treatments (including adjuvant or neoadjuvant regimens if relapse within six months prior to C1D1)
16. Active moderate alcohol consumption, at screening, more than 100/140 grams of alcohol per week for female/male patients, respectively
17. Liver cirrhosis Child-Pugh score B or C
18. Prior treatment with an anti-CTLA-4 or anti-LAG3 in combination with PD-1/PD-L1 CPI, unless discussed and agreed with the Sponsor
19. Prior treatment with SMAC mimetics
20. Prior PD-1/PD-L1 discontinuation due to severe immune-related toxicity, not resolved upon adequate steroids/immunosuppressive treatment
21. Requirement of concomitant treatment with any prohibited medication (See Appendix B: Prohibited medications and special Warnings)
22. Ongoing toxicity from prior administration of any other investigational drug and/or anti-cancer treatment of >Grade 1 NCI-CTCAE v5.0 before treatment start (except for grade 2 alopecia, stable sensory neuropathy, vitiligo or any endocrinopathy adequately managed by replacement hormonal therapy)
23. Patients with known history of:
   a. Uncontrolled or symptomatic angina or myocardial infarction, within the last 12 months prior to C1D1
   b. Elevated (>ULN) troponins (T or I) or creatine phosphokinase (CPK)>1.5×ULN during screening
   c. Symptomatic intestinal sub-occlusion
   d. Infection, active or latent by human immunodeficiency virus (HIV), hepatitis B virus (HBV) and hepatitis C virus (HCV)
   e. Ongoing arrhythmias requiring treatment, including asymptomatic QTc interval as corrected by Fridericia (F)>480 msec
   f. In patients previously treated with anthracycline-containing chemotherapy or thoracic RT, non-adequate cardiac function with left ventricular ejection fraction (LVEF)<50%, measured by an echocardiogram (ECHO) or a multigated acquisition (MUGA) as per institutional standards
   g. Active rheumatoid arthritis, active inflammatory bowel disease (IBD), primary sclerosing cholangitis, autoimmune hepatitis, systemic lupus erythematous (SLE), multiple sclerosis or any other ongoing autoimmune disease requiring systemic treatment (excluding vitiligo, mild cutaneous psoriasis and asymptomatic autoimmune endocrinopathy well controlled under hormonal replacement therapy)
   h. Evidence of active, non-infectious pneumonitis or a history of interstitial lung disease
24. Evidence or clinical suspicion of active bleeding or requirement of red blood cell (RBC) transfusions within 4 weeks prior to C1D1
25. History of allergic reactions attributed to compounds of similar chemical or biologic composition to Debio 1143 or nivolumab or their constituents
26. History of another non-metastatic, low grade malignancy other than the primary tumor within the last 3 years prior to C1D1, except: completely resected non-melanoma skin cancer, second SCCHN tumors, completely resected minimally invasive bladder cancer, completely resected≤pT1N0 breast cancer, low grade prostate cancer if PSA≤10 ng/ml, non-metastatic localized low grade RCC or any previous (non-synchronous) malignancy if related to germ-line mutations linked to either MSI-High-(Lynch syndrome) or BRCA-1/2 hereditary cancer syndromes, for patients included either in cohort 3 or 4. For these indications there are no time restrictions with regards to occurrence prior to C1D1.
27. Pregnant or lactating females
28. Unable to swallow or retain oral medications
29. Known contraindication to contrast-enhanced MRI and CT
30. Patient unwilling or unable to comply with study visits/assessments

In both Part A and Part B, the drugs are administered as follows: Debio 1143 is being administered once daily for 10 consecutive days every 2 weeks (i.e. 10 days on, 4 days off) at a starting dose of 150 mg. Dose increments for subsequent dose groups is 50 mg (i.e. 100, 150, 200, 250 mg). Patients should fast 2 hours before dosing and should fast at least 1 hour post dose. Water is permitted freely. Further dose levels may be considered if pharmacokinetic (“PK”) analysis identifies lower drug exposure of either Debio 1143 or nivolumab.

Nivolumab is administered at 240 mg through an i.v. infusion over at least 30 min, Q2W (i.e. on days 1 and 15 of a 28-day cycle), From Cycle 3, a switch to 480 mg nivolumab through an i.v. infusion over at least 60 min, Q4W (Day 1 of each 28-day cycle), will be allowed on a per-patient basis, based on the Investigator's judgement and Sponsor agreement. The nivolumab dose will not be escalated. Lower doses of nivolumab will not be explored; dose reduction will not be allowed.

Nivolumab is administered in accordance with locally approved labelling. Duration of i.v. infusion is over 30 min for the 240 mg dose or 60 min for the 480 mg dose (−10/+20 minutes, i.e. 50 to 80 minutes). Administration takes place once every 2 weeks, on days 1 and 15 of each cycle for the 240 mg dose or once every 4 weeks, on day 1 of each cycle for the 480 mg dose. A manual of preparation describes in detail infusion bags and medical devices to be used for the preparation of the dilution and subsequent administration.

Premedication with an antihistamine (anti-H1) and with acetaminophen (paracetamol) approximately 30 to 60 minutes prior to each dose of nivolumab is optional (e.g. 25-50 mg diphenhydramine and 500-650 mg acetaminophen (paracetamol) i.v. or oral equivalent). This regimen may be modified based on local treatment standards and guidelines, as appropriate. Premedication should be administered for subsequent nivolumab doses based upon clinical judgment and presence/severity of prior infusion reactions.

The primary endpoint of the first aspect of this study is the recommended phase II dose (RP2D) of Debio 1143 when combined with the standard dose of nivolumab, in patients with advanced solid malignancies who received prior systemic standard treatment and failed a prior PD-1/PD-L1-containing treatment, as per dose limiting toxicity (DLT) occurrence in less than one-third of evaluable treated patients at the RP2D dose level. DLT is defined as any of the following Adverse

Events (AEs) during the first treatment cycle (i.e. 4 weeks or longer in case of dosing delays) if deemed related to treatment:
Any Grade 4 or 5 hematologic toxicity, clinical or laboratory non-hematologic toxicity
Febrile neutropenia any grade, Grade 3 thrombocytopenia if associated with bleeding or requiring platelet transfusion
Grade 2 or higher cardiac or neurologic toxicities, pneumonitis that does not resolve within 3 days after starting steroids at adequate doses or that was recurrent upon steroid tapering or withdrawal, myasthenia gravis, myositis, polymyositis, Guillain-Barre syndrome (excluding peripheral purely-sensory neuropathies)
Grade 2 or higher alanine transaminase (ALT)/aspartate transaminase (AST) increase with a concurrent increase of total bilirubin>2×upper limit of normal (ULN) and concurrent with alkaline phosphatase (ALP)≤2 ×ULN (Hy's law), in the absence of objective causes of biliary obstruction due to obvious disease-related causes
Any other Grade 3 non-hematologic, treatment-related clinical toxicity lasting 3 or more days despite optimal supportive care, or requiring admission for appropriate medical management; hypersensitivity and/or infusion-related reactions are excluded if not reoccurring after appropriate pre-medication and/or prolonging infusion time
Grade 3 non-hematologic laboratory value (excluding lipase, amylase and/or autoimmune endocrinopathies manageable by replacement therapies) if:
   a. Symptomatic and
   b. Medical intervention was promptly required to treat the patient, or
   c. The abnormality led to hospitalization or
   d. The abnormality did not resolve to at least Grade 1 within 2 weeks (including the steroid tapering period) despite adequate medical intervention/treatment
A delay of >2 weeks due to drug-related toxicity in initiating Cycle 2
Unable to complete at least 70% of the scheduled treatment, i.e. more than six Debio 1143 skipped doses in Cycle 1 due to treatment-related toxicity
Required dose reduction in Cycle 1 or on Cycle 2 Day 1 or requirement for treatment withdrawal due to treatment-related toxicity (even if not meeting other DLT criteria)

The primary endpoint of the second aspect of this study is confirmed objective response rate (ORR) as per RECIST v1.1 and/or GCIG criteria (patients with platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD), if applicable).

The secondary endpoints of this study are:
Incidence and severity of treatment-emergent AEs and laboratory abnormalities, graded according to NCI-CTCAE version 5.0 criteria;
Changes in vital signs: systolic/diastolic blood pressure, heart rate (both after at least 5 minutes of supine rest), temperature, and weight; ECG and ECOG-PS
Incidence of premature treatment discontinuations and treatment modifications due to AEs and laboratory abnormalities (i.e., treatment compliance)
Tumor response determined according to RECIST v1.1 and/or GCIG criteria (patients with platinum-resistant epithelial ovarian cancer (EOC), endometrial cancer, primary peritoneal cancer (PPC) and cervical cancer, with known MSI-H/ MMRd, hereditary/somatic mutations of the BRCA1 and BRCA2 genes or other DNA DDR abnormalities (incl. HRD), if applicable):
   Confirmed (Part A) and unconfirmed (Parts A and B)ORR
   Disease control rate (DCR), defined as any response, partial or complete (PR or CR)+stable disease (SD)
   Time-related endpoints as median time to response, median DOR, median PFS, PFS rate at 6, 12 and 18 months, median OS, OS rate at 12 months and long-term OS (defined as OS measured as long as at least 80% of patients included in a given cohort are still followed)
PK parameters of Debio 1143 and Debio 1143-MET1 as defined in the available population PK model (Rouits E, Csajka C. Debio 1143 population pharmacokinetic analysis. Debiopharm studies Debio 1143-101, Debio 1143-102 and Debio 1143-103.; 2016) and, if appropriate, post-hoc estimates of areas under the curve (AUCs), Cmax, and Cmin; serum concentration versus time profiles of nivolumab and, if deemed appropriate, relevant nivolumab PK parameters derived from a population PK model. (Bajaj G, Wang X, Agrawal S, Gupta M, Roy A, Feng Y. Model-Based Population Pharmacokinetic Analysis of Nivolumab in Patients With Solid Tumors. CPT Pharmacometrics Syst Pharmacol. 2017; 6(1):58-66)

It is to be appreciated that the Detailed Description section, and not the Summary and Abstract sections, is intended to be used to interpret the claims. The Summary and Abstract sections sets forth one or more, but not all, exemplary embodiments of the present invention as contemplated by the inventor(s), and thus, are not intended to limit the present invention and the appended claims in any way.

The present invention has been described above with the aid of functional building blocks illustrating the implementation of specified functions and relationships thereof. The boundaries of these functional building blocks have been arbitrarily defined herein for the convenience of the description. Alternate boundaries can be defined so long as the specified functions and relationships thereof are appropriately performed.

The foregoing description of the specific embodiments will so fully reveal the general nature of the invention that others can, by applying knowledge within the skill of the art, readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance.

The breadth and scope of the present invention should not be limited by any of the above-described exemplary embodiments, but should be defined only in accordance with the following claims and their equivalents.

1. A combination product comprising:
(i) Debio 1143 or another IAP antagonist; and
(ii) nivolumab.

2. The combination product according to claim 1, wherein the combination product is a pharmaceutical combination product and further comprises a pharmaceutically acceptable carrier, diluent, excipient and/or adjuvant.

3. A pharmaceutical composition comprising:
(i) Debio 1143 or another IAP antagonist;
(ii) nivolumab; and
(iii) a pharmaceutically acceptable carrier, diluent, excipient and/or adjuvant.

4. The combination product according to any one of claims 1-2 or the pharmaceutical composition according to claim 3, wherein nivolumab and Debio 1143, or another IAP antagonist, are provided in a single or separate unit dosage forms.

5. The combination product according to any one of claim 1-2 or 4 or the pharmaceutical composition according to any one of claims 3-4, for use as a medicament.

6. The combination product according to any one of claim 1-2 or 4-5, or the pharmaceutical composition according to any one of claims 3-5, for use in a method of treating cancer; wherein, optionally, the cancer is advanced, unresectable and/or metastatic solid malignancy.

7. The combination product or pharmaceutical composition for use according to claim 6, wherein the cancer is selected from the group consisting of small cell lung cancer (SCLC); squamous cell carcinoma of the head and neck (SCCHN); GI cancers, including esophageal, gastric, colorectal or pancreatobiliary tumors, with known known microsatellite instability-high (MSI-H), mismatch repair deficiency (MMRd) or other known DNA damage repair (DDR) abnormalities, including homologous recombination deficiency (HRD) in gastrointestinal (GI) cancers; platinum-resistant epithelial ovarian cancer (EOC), endometrial cancer, primary peritoneal cancer (PPC) and cervical cancer, with known MSI-H/MMRd, hereditary/somatic mutations of the BRCA1 and BRCA2 genes or other DNA DDR abnormalities (incl. HRD).

8. The combination product or pharmaceutical composition for use according to any one of claims 6-7, wherein the method comprises administering about 75 to about 250 mg per day of Debio 1143 and about 240 mg every 14 days of nivolumab, preferably 100 mg, 150 mg or 200 mg per day of Debio 1143 and 240 mg every 14 days of nivolumab.

9. The combination product or pharmaceutical composition for use according to any one of claims 6-8, wherein the method of treatment comprises a 28-day cycle comprising
(a) administering Debio 1143 for a first 10 consecutive day period;
(b) administering no Debio 1143 for a first 4 consecutive day period;
(c) administering Debio 1143 for a second 10 consecutive day period;
(d) administering no Debio 1143 for a second 4 consecutive day period;
(e) administering the nivolumab on day 1 of the 28-day cycle; and
(f) administering the nivolumab on day 15 of the 28-day cycle.

10. The combination product or pharmaceutical composition for use according to any one of claims 6-9, wherein a patient, who is administered the combination product or pharmaceutical composition, underwent at least one round of prior cancer therapy;
wherein, optionally, the cancer was resistant or became resistant to prior therapy.

11. The combination product or pharmaceutical composition for use according to any one of claims 9-13, wherein the patient who is administered the combination product or pharmaceutical composition has previously received platinum-based therapy, preferably the patient has relapsed or progressed after receiving the platinum-based therapy.

12. A kit comprising nivolumab and Debio 1143, or another IAP antagonist, and a package insert comprising instructions for using nivolumab and Debio 1143, or another IAP antagonist, to treat or delay progression of a cancer in a patient; wherein, optionally,
the kit comprises a first container, a second container and a package insert, wherein the first container comprises at least one dose of a medicament comprising nivolumab, the second container comprises at least one dose of a medicament comprising Debio 1143, or another IAP antagonist, and the package insert comprises instructions for treating a subject for cancer using the medicaments.

13. A composition comprising nivolumab for use in a method of treating cancer, wherein the composition is to be administered in combination with Debio 1143, or another IAP antagonist.

14. A composition comprising Debio 1143, or another IAP antagonist, for use in a method of treating cancer, wherein the composition is to be administered in combination with nivolumab.
